(19) United States
(12) Patent Application Publication
(10) Pub. No.: US 2025/0009810 A1
(43) Pub. Date: Jan. 9, 2025

(54) THERAPEUTIC TREATMENT OF SKIN DISEASE WITH RECOMBINANT COMMENSAL SKIN MICROORGANISMS

(71) Applicant: Azitra Inc., Branford, CT (US)

(72) Inventors: Azim Momin Munivar, New Haven, CT (US); Travis Michael Whitfill, Dallas, TX (US)

(21) Appl. No.: 18/735,645

(22) Filed: Jun. 6, 2024

Related U.S. Application Data
(63) Continuation of application No. 16/890,552, filed on Jun. 2, 2020, now Pat. No. 12,036,248.
Application No. 16/890,552 is a continuation of application No. 15/312,441, filed on Nov. 18, 2016, now Pat. No. 10,702,558.
(60) Provisional application No. 62/005,558, filed on May 30, 2014.
(60) Provisional application No. 62/005,652, filed on May 30, 2014.

(51) Int. Cl. A61K 35/74; A61K 9/00; A61K 38/17; A61P 17/00; C07K 14/47; C12N 1/20; C12N 15/74

(52) U.S. Cl. CPC A61K 35/74; A61K 9/0014; A61K 38/1709; A61P 17/00; C07K 14/4713; C12N 1/20; C12N 15/74; C07K 2319/02; C07K 2319/10

(57) ABSTRACT

The invention relates to methods for treating or preventing abnormal skin conditions in a human in need thereof, comprising administering a cell culture composition comprising a living culture of bacteria comprising at least one engineered strain that produces a recombinant polypeptide for therapeutic treatment of the abnormal skin condition. The invention also relates to pharmaceutical compositions containing, as the active principle, engineered microorganisms expressing non-vaccinogenic pharmacologically active recombinant therapeutic polypeptides in order to treat or prevent abnormal skin conditions.

### RELATED APPLICATIONS

This application is a continuation of U.S. patent application Ser. No. 16/890,552 filed on Jun. 2, 2020, now U.S. Pat. No. 12,036,248, which is a continuation of U.S. patent application Ser. No. 15/312,441 filed on Nov. 18, 2016, now U.S. Pat. No. 10,702,558, which is a National Phase application claiming the benefit of priority under 35 U.S.C. § 371 to Patent Convention Treaty (PCT) International Application Serial No. PCT/US2015/032972 filed May 28, 2015, which claims priority to U.S. Provisional Patent Application No. 62/005,558 filed on May 30, 2014 and U.S. Provisional Patent Application No. 62/005,652 filed on May 30, 2014, the disclosures of each of which are incorporated by reference herein in their entirety.

### SEQUENCE LISTING

The application contains a Sequence Listing which has been submitted electronically in .XML format and is hereby incorporated by reference in its entirety. Said .XML copy, created on Jul. 1, 2022, is named “129062-00603_SL.xml” and is 69,644 bytes in size. The sequence listing contained in this .XML file is part of the specification and is hereby incorporated by reference herein in its entirety.

### FIELD OF THE INVENTION

The present invention relates generally to the fields of synthetic biology, microbiology, protein chemistry, skin care, extracellular protein transport, intracellular transport, and the treatment and prevention of skin disease. More specifically, it concerns methods and compositions employing engineered strains of commensal skin bacteria that produce recombinant polypeptides for the therapeutic treatment and prevention of skin diseases, in particular employing an engineered strain of Staphylococcus epidermidis that produces recombinant filaggrin.

### BACKGROUND OF THE INVENTION

The advent of compounds that are generated using recombinant DNA technology has facilitated development of a vast array of therapeutic agents having the potential to treat a great variety of disease states in animals, in particular in humans. These agents are primarily protein in nature.

The epidermis, the squamous stratified epithelium of the skin, consists of multiple sublayers and is one of the most important barriers of the body against the outside world. The stratum corneum is the outermost layer of the epidermis and develops as a result of the final anucleated step in keratinocyte differentiation from the cells in nucleated epidermal layers. Although the stratum corneum is recognized as the most important physical barrier, the nucleated epidermal layers are also significant in barrier function (Proksch, Brandner et al. 2008). Together, the skin barrier protects against extensive water loss in one direction (inside-outside barrier) and against the invasion of harmful substances from the environment (outside-inside barrier) (Proksch, Brandner et al. 2008). The maintenance of the barrier is also important for balanced proliferation in the basal layer and preservation of the calcium ion gradient and thus proper epidermal differentiation (Lee, Jeong et al. 2006).

Recent work suggests that skin commensal microorganisms are essential to maintaining healthy skin and maintaining the skin barrier. Commensal microorganisms are ones that are beneficial to their subject. e.g., human, hosts. Studies also suggest that certain skin diseases (such as acne vulgaris and atopic dermatitis) can be associated with disruptions to the normal microflora (Lin, Wang et al. 2007). Therefore, the idea that the skin microflora can be modulated using specific skin commensals to promote health or inhibit disease has received some attention (Muizzuddin, Maher et al. 2012). Many skin commensal bacteria are not considered pathogenic, therefore, these bacteria can potentially be used topically if they have therapeutic value (Nakatsuji and Gallo 2014). So far the limited amount of research in this area suggests that conventional probiotic bacteria can be of significant value when used on the skin. For example, topical application of a Bifidobacterium longum lysate has been shown to induce clinical improvement of “reactive skin” (Gueniche, Bastien et al. 2010). This is skin that is more sensitive to physical changes such as atmospheric temperature, and to chemical changes such as seen with topically applied products (Gueniche, Bastien et al. 2010). Application of the B. longum lysate to the skin of volunteers was shown to decrease sensitivity and decrease transepidermal water loss (TEWL) after tape stripping. Additionally, application of the lysate to ex vivo skin was shown to decrease signs of inflammation such as vasodilation, edema and TNF-α release (Gueniche, Bastien et al. 2010, Nakatsuji and Gallo 2014, Volz, Skabytska et al. 2014). Topical application of Lactobacillus plantarum has also been demonstrated to improve tissue repair in a burned mouse model and prevent infection in chronic leg ulcers and burns in humans (Peral, Martinez et al. 2009. Peral, Rachid et al. 2010, Brachkova, Marques et al. 2011).

A number of current limitations exist in the treatment of skin, however. Many treatments, such as topical corticosteroids or expensive biologics, do not treat the underlying issues of deficient intrinsic protein in the epidermis or imbalances in the microbial diversity in the skin. While recombinant proteins represent a promising group of therapeutic agents in the treatment of skin disease, several problems accompany their use in the context. Traditional methods employ the use of purification of recombinant proteins that are extracted from bacterial systems, purified, concentrated, and incorporated into a delivery system. The purification of recombinant proteins is often a very costly method of obtaining protein. Moreover, a number of issues accompany this, including proteolytic degradation, inefficient delivery, and the need for repeated application overtime to achieve therapeutic effect.

Our method, in contrast, addresses these issues by allowing bacteria to colonize the skin and continually secrete therapeutic polypeptides of benefit to the skin. This approach reduces need for multiple topical applications by creating a sustainable delivery system for therapeutic proteins.

Most of the microorganisms until now used for the production of recombinant proteins cannot be in safely administered to humans and animals, not being usual components of the physiological flora or being devoid of any pathogenic risk. This is particularly true for Escherichia coli, which is considered pathogenic in many cases. However, here, we describe methods of production of recombinant proteins using microorganisms that can be safety administered to humans and animals, particularly Staphylococcus epidermidis.

Other species, already used for the production of recombinant proteins, can also be used provided they meet the requisites of non-pathogenicity and ability to colonize human or animal mucosae. For example, Bacillus subtilis has been widely used as a cloning vector for producing a large number of eukaryotic proteins in view of its recognized advantageous properties (Simonen and Palva 1993). The present invention allows therefore, by suitably selecting and adapting the microorganism, the polypeptide to be expressed and the expression vectors, previously used for the production in laboratory or industrial environment, to address specific skin diseases (see FIG. 1). The present invention concerns therefore the therapeutic use of said engineered microorganisms and compositions containing the same, thereby satisfying a long felt need in the area of therapeutic delivery of drugs to treat skin conditions in humans.

Atopic dermatitis (AD), which is also known as eczema, is a chronic inflammatory skin disease that usually involves a defect in the stratum corneum of the skin, which is the

protective layer comprising the outermost part of the skin. This defect is caused by a confluence of genetic, environmental, and immunological factors and is associated with an overgrowth of Staphylococcus aureus. The genetic basis of AD appears to involve a defect in the filaggrin gene (FLG), seen in approximately 50% of AD patients (McAleer 2013). The prevalence of atopic dermatitis is increasing, and affects 0.1-1% of the
population worldwide, with higher rates in developed countries. AD is more common in children, affecting as many as 20% of children (McAleer 2013). Of these children, 25% never recover and continue to suffer from atopic dermatitis into adulthood. In many cases, AD can lead to more severe allergic conditions, including asthma, through a process known as the “atopic march.”

The pathogenesis of AD is not completely understood, but defects in filaggrin expression are attributed to most cases of atopic dermatitis, and the pathogenesis is related to an immune response. It has been shown that AD can be caused by a combination of dry skin, skin that is prone to itching more than the average person, infections caused by bacteria, viruses, fungi, etc., and emotional and environmental factors. Filaggrin is encoded by the

FLG gene on the 1q21 epidermal differentiation complex. Filaggrin is a protein produced by differentiating keratinocytes, and functions to aggregate keratin filaments into a cytoskeleton that, in combination with other components, comprises the cornified cell envelope (Brown & McLean 2012). Filaggrin loss of function mutations (R501X and 2282del4) cause ichthyosis vulgaris, the most common inherited disorder of keratinization. The same mutations also are associated with other skin and allergic conditions including AD, irritant contact dermatitis, asthma, and food allergy. Several studies have shown that increasing filaggrin on the skin helps to mitigate the AD phenotype (e.g. Stout et al. 2014, Otsuka et al. 2014).

Currently, the major therapies for AD include topical corticosteroids and antibiotics, both of which have limited efficacy in more severe cases of AD. Major drawbacks of antibiotics include issues of antibiotic resistance and dysregulation of the microbiome ecology. The microbiome is the aggregate of microorganisms present in a particular environment. The microbiome mentioned herein, is the totality of the microorganisms present on the largest human organ, the skin. Topical corticosteroids provide respite from symptoms for mild AD patients, but do not address the needs of those with chronic or severe disease. While some targeted therapies for AD treatment are under development, the competitive landscape in this market is characterized by a largely unmet need foran effective, targeted therapy for AD. One biologic therapy, Dupilumab, is an anti-IL4/13-receptor monoclonal antibody that is under Phase II clinical trial for AD, being developed by Regeneron Pharmaceuticals and Sanofi. This drug has shown promising efficacy, and is expected to reach markets in the next five years. Other similar biologic therapeutics for immunomodulation of AD are under development. However, these drugs are expected to cost tens of thousands of dollars for consumers due to high production and R&D costs, and insurance companies may not necessarily cover these costs. While these drugs attenuate the immune response, they do not have direct effects on re-establishing the skin barrier and normalizing the microbial ecology to prevent S. aureus infections. Thus, new compositions and methods for the effective treatment and prevention of abnormal skin conditions such as AD, while minimizing unwanted side effects are required.

### BRIEF DESCRIPTION OF THE FIGURES

FIG. 1 depicts a flowchart of the present invention, demonstrating the creation of an engineered bacterial strain that produces a recombinant polypeptide for therapeutic treatment or prevention of an abnormal skin condition.

FIG. 2 depicts a vector design of the plasmid and shuttle vector pBT-2. VectorpBT-2 is exemplary of alternative shuttle vectors that can be used for cloning in E. coli and expression in Staphylococcus.

FIG. 3 depicts a vector design of a modified version of the shuttle vector pBT-2 including the FLG (filaggrin) gene insert, described in FIG. 4. This construct is exemplary of alternative constructs for filaggrin expression in S. epidermidis and has previously been described as pAZT-01.

FIG. 4 is a detailed map of the FLG gene insert that was cloned into the plasmid pBT-2, shown previously in FIG. 2, and can be cloned into various vectors for expression in Staphylococcus. This insert includes xylose repressor (xylR), xylose operator (xylO), xylose isomerase gene (xylA) including the cis-acting catabolite-responsive element (CRE), the Sec (secretion pathway) export signal, gene encoding filaggrin (FLG), and the cell penetrating peptide.

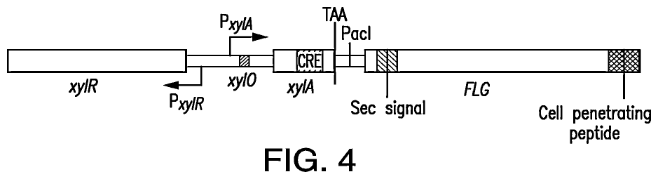

FIG. 5 depicts a vector design of the plasmid pJB38 (Cheung, AL., et al. (2004)). Restriction sites are built into the multiple cloning site (MCS) of the vector. Inserted into the MCS of the vector is a filaggrin expression vector with a xylose-inducible promoter, ribosome binding site, export signal (for example SecA export signal), partial FLG (filaggrin) repeats flanking the filaggrin monomer, and a cell penetrating signal. The insert of pAZT-01 was constructed and cloned into pJB38 by Bio Basic, Inc, (Markham, ON, Canada).

FIG. 6A and FIG. 6B depict the transformation of S. epidermidis ATCC12228 with pCM11 containing a GFP (green fluorescent protein) expression platform as verified by fluorescence microscopy. Plasmid pCM11 (which contains a GFP expression platform) was used to transform E. coli DH5α cells using a heat shock method. Plasmid pCM11 isolated from E. coli DH5α was then used for transformation of E. coli DC10B using electroporation. Plasmid pCM11 was then isolated from E. coli DC10B and used to transform S. epidermidis using electroporation. As a control, FIG. 6A demonstrates the inherent fluorescence of S. epidermidis, confirming that the strain has minimal to no inherent fluorescence by itself prior to transformation with a GFP expressing plasmid. In FIG. 6B, S. epidermidis is transformed with pCM11 containing the GFP insert, which is in turn expressed, as demonstrated by the fluorescence. These images confirm the transformation of S. epidermidis and the expression of pCM11 within that strain, as indicated by the GFP fluorescence.

FIG. 7A and FIG. 7B depict S. epidermidis ATCC12228 transformed with pCM11 containing the GFP (green fluorescent protein) insert, which was in turn applied on the dorsal skin of mice for three days. Sections of the skin were then taken post-mortem for light microscopy (FIGS. 7A & 7B). Sections shown here were stained using Malachite Green stain and visualized at 200× (FIG. 7A) and further expansion (FIG. 7B). This figure demonstrates the colonization of S. epidermidis on the mouse skin after three days. (Staph epi refers to Staphylococcus epidermidis).

FIG. 8A, FIG. 8B, and FIG. 8C depict S. epidermidis ATCC12228 transformed with pCM11 containing the GFP (green fluorescent protein) insert, which was in turn applied on the dorsal skin of mice for three days. Sections of the skin were then taken post-mortem for light microscopy (FIGS. 8A & 8B). FIG. 8A shows fluorescence of GFP indicating not only that S. epidermidis colonizes the skin after three days, but also that expression of GFP is maintained at that time. FIG. 8A shows fluorescence from GFP while FIG. 8B shows a section of skin stained with both DAPI (4′,6-diamidino-2-phenylindole) and an anti-GFP monoclonal antibody. FIG. 8C demonstrates a section of the skin visualized using two-photon microscopy, demonstrating the extent of S. epidermidis colonization and GFP expression, as indicated by the fluorescence.

### SUMMARY OF THE INVENTION

The invention refers to pharmaceutical compositions containing, as the active principle, engineered microorganisms expressing non-vaccinogenic, pharmacologically active recombinant therapeutic polypeptides (i.e. proteins, peptides, or amino acids) in order to treat skin conditions. Such non-vaccinogenic polypeptides, in contrast to those employed in vaccines, are those that generally do not incite an immune response and yet are therapeutically effective.

The present invention relates to a method of treating or preventing abnormal skin conditions in a patient (human or animal subject) in need thereof, the method comprising administering to said patient a cell culture composition comprising a bacterial strain selected from Bifidobacterium, Brevibacterium, Propionibacterium, Lactococcus, Streptococcus, Staphylococcus (e.g., S. epidermidis), Lactobacillus (e.g., L. acidophilus), Enterococcus, Pediococcus, Leuconostoc, or Oenococcus, and mixtures thereof, wherein the composition comprises at least one engineered bacterial strain that produces a therapeutically effective amount of a recombinant polypeptide for therapeutic treatment or prevention of the abnormal skin condition.

In one aspect the present invention relates to a method wherein said composition comprises bacterial strain containing human target DNA recombined with bacterial DNA to make an engineered bacterial strain.

In another aspect the present invention relates to a method wherein the composition comprises an engineered strain of Staphylococcus epidermidis.

In another aspect the present invention relates to a method wherein the cell culture composition is a living cell culture composition.

In another aspect the present invention relates to a method wherein the skin condition is selected from psoriasis, acne, atopic dermatitis, allergic contact dermatitis, epidermolytic hyperkeratosis, seborrheic dermatitis, eczema, dry skin, allergy, rashes, UV-irritated skin, detergent irritated skin (including irritation caused by enzymes and molecules used in washing detergents and sodium lauryl sulfate), thinning skin (e.g. skin from the elderly and children), bullous pemphigoid, pemphigus vulgaris, impetigo, vitiligio, baldness, and hirsutism.

In another aspect the present invention relates to a method wherein said skin condition is eczema.

In another aspect the present invention relates to a method wherein said eczema is a mild eczema.

In another aspect the present invention relates to a method wherein said eczema is a moderate eczema.

In another aspect the present invention relates to a method wherein said eczema is a severe eczema.

In another aspect the present invention relates to a method wherein said skin condition is hand eczema.

In another aspect the present invention relates to a method of treating or preventing abnormal skin conditions in a patient (human or animal subject) in need thereof, the method comprising administering to said patient a cell culture composition comprising a bacterial strain selected from Bifidobacterium, Brevibacterium, Propionibacterium, Lactococcus, Streptococcus, Staphylococcus (e.g., S. epidermidis), Lactobacillus (e.g., L. acidophilus), Enterococcus, Pediococcus, Leuconostoc, or Oenococcus, and mixtures thereof wherein the cell culture composition comprises at least one engineered bacterial strain that produces a therapeutically effective amount of a recombinant polypeptide comprising (a) a filaggrin amino acid sequence, (b) a secretion signal, and (c) a cell penetrating peptide for therapeutic treatment or prevention of the abnormal skin condition.

In another aspect the present invention relates to a method wherein the cell culture composition comprises Staphylococcus epidermidis.

In another aspect the present invention relates to a method wherein the cell culture composition is a living culture composition.

In another aspect the present invention relates to a method wherein the filaggrin amino acid sequence comprises a sequence selected from SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, or SEQ ID NO 17, and combinations thereof.

In another aspect the present invention relates to a method wherein the filaggrin amino acid sequence comprises SEQ ID NO 1.

In another aspect the present invention relates to a method wherein the filaggrin amino acid sequence has at least about 75% homology to SEQ ID NO 1.

In another aspect the present invention relates to a method wherein the filaggrin amino acid sequence has at least about 80% homology to SEQ ID NO 1.

In another aspect the present invention relates to a method wherein the filaggrin amino acid sequence has at least about 85% homology to SEQ ID NO 1.

In another aspect the present invention relates to a method wherein the filaggrin amino acid sequence has at least about 90% homology to SEQ ID NO 1.

In another aspect the present invention relates to a method wherein the filaggrin amino acid sequence has at least about 95% homology to SEQ ID NO 1.

In another aspect the present invention relates to a method wherein the filaggrin amino acid sequence has at least about 75% homology, or 80% homology, or 85% homology, or 90% homology, or 95% homology to a sequence selected from SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, or SEQ ID NO 17.

In another aspect the present invention relates to a method wherein the skin condition is selected from psoriasis, acne, atopic dermatitis, allergic contact dermatitis, epidermolytic hyperkeratosis, seborrheic dermatitis, eczema, dry skin, allergy, rashes, UV-irritated skin, detergent irritated skin (including irritation caused by enzymes and molecules used in washing detergents and sodium lauryl sulfate), thinning skin (e.g. skin from the elderly and children), bullous pemphigoid, pemphigus vulgaris, impetigo, vitiligio, baldness, and hirsutism.

In another aspect the present invention relates to a method wherein the skin condition is eczema.

In another aspect the present invention relates to a pharmaceutical composition for treating or preventing abnormal skin conditions in a patient (human or animal subject) in need thereof, comprising (a) a cell culture composition comprising a bacterial strain selected from Bifidobacterium, Brevibacterium, Propionibacterium, Lactococcus, Streptococcus, Staphylococcus (e.g., S. epidermidis), Lactobacillus (e.g., L. acidophilus), Enterococcus, Pediococcus, Leuconostoc, or Oenococcus, and mixtures thereof, wherein the cell culture composition comprises at least one engineered bacterial strain that produces a therapeutically effective amount of a recombinant polypeptide for therapeutic treatment or prevention of abnormal skin conditions and (b) a pharmaceutically acceptable carrier.

In another aspect the present invention relates to a pharmaceutical composition wherein said composition comprises a bacterial strain containing human target DNA recombined with bacterial DNA.

In another aspect the present invention relates to a pharmaceutical composition wherein said composition comprises an engineered strain of Staphylococcus epidermidis.

In another aspect the present invention relates to a pharmaceutical composition wherein said composition comprises a living cell culture composition.

In another aspect the present invention relates to a pharmaceutical composition wherein said composition comprises 0% water to no more than about 90% water.

In another aspect the present invention relates to a pharmaceutically acceptable carrier that is selected from an aqueous solution, an emulsion, a cream, a lotion, a gel, or an ointment.

In another aspect the present invention relates to a pharmaceutical composition for treating or preventing abnormal skin conditions in a patient (human or animal subject) in need thereof, comprising (I) a cell culture composition comprising a bacterial strain selected from Bifidobacterium, Brevibacterium, Propionibacterium, Lactococcus, Streptococcus, Staphylococcus (e.g., S. epidermidis), Lactobacillus (e.g., L. acidophilus), Enterococcus, Pediococcus, Leuconostoc, or Oenococcus, and mixtures thereof, wherein the cell culture composition comprises at least one engineered bacterial strain that produces a therapeutically effective amount of a recombinant polypeptide comprising (a) a filaggrin amino acid sequence, (b) a secretion signal, and (c) a cell penetrating peptide and (II) a pharmaceutically acceptable carrier for therapeutic treatment or prevention of the abnormal skin condition.

In another aspect the present invention relates to a pharmaceutical composition wherein the cell culture composition comprises a bacterial strain containing human target DNA recombined with bacterial DNA.

In another aspect the present invention relates to a pharmaceutical composition wherein the cell culture composition comprises Staphylococcus epidermidis.

In another aspect the present invention relates to a pharmaceutical composition wherein the cell culture composition is a living cell culture composition.

In another aspect the present invention relates to a pharmaceutical composition wherein the filaggrin amino acid sequence comprises a sequence selected from SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, or SEQ ID NO 17, and combinations thereof.

In another aspect the present invention relates to a pharmaceutical composition wherein the filaggrin amino acid sequence comprises SEQ ID NO: 1.

In another aspect the present invention relates to a pharmaceutical composition wherein the filaggrin amino acid sequence has at least about 75% homology to SEQ ID NO 1.

In another aspect the present invention relates to a pharmaceutical composition wherein the filaggrin amino acid sequence has at least about 80% homology to SEQ ID NO 1.

In another aspect the present invention relates to a pharmaceutical composition wherein the filaggrin amino acid sequence has at least about 85% homology to SEQ ID NO 1.

In another aspect the present invention relates to a pharmaceutical composition wherein the filaggrin amino acid sequence has at least about 90% homology to SEQ ID NO 1.

In another aspect the present invention relates to a pharmaceutical composition wherein the filaggrin amino acid sequence has at least about 95% homology to SEQ ID NO 1.

In another aspect the present invention relates to a pharmaceutical composition wherein the filaggrin amino acid sequence has at least about 75% homology, or 80% homology, or 85% homology, or 90% homology, or 95% homology to a sequence selected from SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, or SEQ ID NO 17.

In another aspect the present invention relates to a pharmaceutical composition wherein the composition comprises 0% water to no more than about 90% water.

In another aspect the present invention relates to a pharmaceutical composition wherein the pharmaceutically acceptable carrier is an aqueous solution, an emulsion, a cream, a lotion, a gel, or an ointment.

In another aspect the present invention relates to use of a cell culture composition for the manufacture of a medicament for treating or preventing abnormal skin conditions in a patient (human or animal subject) in need thereof, said cell culture composition comprising a bacterial strain selected from Bifidobacterium, Brevibacterium, Propionibacterium, Lactococcus, Streptococcus, Staphylococcus (e.g., S. epidermidis), Lactobacillus (e.g., L. acidophilus), Enterococcus, Pediococcus, Leuconostoc, or Oenococcus, and mixtures thereof wherein the cell culture composition comprises at least one engineered bacterial strain that produces a therapeutically effective amount of a recombinant polypeptide for treating or preventing the abnormal skin condition.

In another aspect the present invention relates to use of a cell culture composition for the manufacture of a medicament for treating or preventing abnormal skin conditions in a patient (human or animal subject) in need thereof, said cell culture composition comprising a bacterial strain selected from Bifidobacterium, Brevibacterium, Propionibacterium, Lactococcus, Streptococcus, Staphylococcus (e.g., S. epidermidis), Lactobacillus (e.g., L. acidophilus), Enterococcus, Pediococcus, Leuconostoc, or Oenococcus, and mixtures thereof wherein the cell culture composition comprises at least one engineered bacterial strain that produces a therapeutically effective amount of a recombinant polypeptide comprising (a) a filaggrin amino acid sequence, (b) a secretion signal, and (c) a cell penetrating peptide for therapeutic treatment or prevention of the abnormal skin condition.

### DEFINITIONS

As used herein, the following terms have the following meanings unless expressly stated to the contrary:

As used herein, the term “abnormal skin condition” refers to a skin state or condition that is generally undesirable or deleterious compared to the normal or baseline condition of human skin. Examples of abnormal skin conditions include: psoriasis, acne, atopic dermatitis, allergic contact dermatitis, epidermolytic hyperkeratosis, seborrheic dermatitis, eczema, dry skin, allergy, rashes, UV-irritated skin, detergent irritated skin (including irritation caused by enzymes and molecules used in washing detergents and sodium lauryl sulfate), thinning skin (e.g. skin from the elderly and children), bullous pemphigoid, pemphigus vulgaris, impetigo, vitiligio, baldness, and hirsutism.

As used herein, the terms “patient” or “subject”, refers to a human or animal (in the case of an animal, more typically a mammal such as domesticated mammals, or animals such as poultry animals and fish and other seafood or freshwater food creatures), that would be subjected to the treatments and compositions of the present invention. Such patient or subject would be considered to be in need of the pharmaceutical compositions of the present invention or of the methods of treating, preventing, or reducing the risk of an abnormal skin condition.

As used herein, the term “therapeutically effective amount” refers to an amount of a pharmaceutical active compound, or a combination of compounds, or an amount of pharmaceutical active compound delivered by an engineered bacterial strain or strains, for example a skin treatment agent or agents, when administered alone or in combination, to treat, prevent, or reduce the risk of a disease state or condition, for example an abnormal skin condition. The term also refers to an amount of a pharmaceutical composition containing an active compound or combination of compounds or an engineered bacterial strain or strains that delivers a pharmaceutical active compound. For example, an effective amount refers to an amount of the compound or an amount of the compound delivered by an engineered bacterial strain or strains present in a formulation given to a recipient patient or subject sufficient to elicit biological activity, for example, activity for treating or preventing an abnormal skin condition.

As used herein, the phrase “pharmaceutically acceptable” refers to those active compounds, materials, engineered bacterial strain or strains, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

As used herein, the term “treating” refers to providing a therapeutic intervention to cure or ameliorate an abnormal skin condition.

As used herein, the term “preventing”, refers to completely or almost completely stopping an abnormal skin condition from occurring, for example when the patient or subject is predisposed to an abnormal skin condition or at risk of contracting an abnormal skin condition. Preventing can also include inhibiting, i.e. arresting the development, of an abnormal skin condition.

As used herein, the term “reducing the risk of”, refers to lowering the likelihood or probability of an abnormal skin condition from occurring, for example when the patient or subject is predisposed to an abnormal skin condition or at risk of contracting an abnormal skin condition.

As used herein, the term “engineered bacterial strain,” refers to a strain of bacteria that has been “genetically modified” or “engineered” by the introduction of DNA prepared outside the organism into the bacterial strain. For example, the introduction of plasmid DNA containing new genes into bacteria will allow the bacteria to express those genes. Alternatively, the DNA containing new genes can be introduced to the bacteria and then integrated into the bacteria's genome, where the bacteria will express those genes.

As used herein, the terms “carriers”, “carrier system” or “vehicles” refer to compatible substances that are suitable for delivering, containing, or “carrying” a pharmaceutical active ingredient or other materials for administration in a topically applied composition to a patient or subject. Carriers useful herein should be pharmaceutically acceptable. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue. Further examples of “carriers” include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

As used herein, the terms “polypeptide” or “protein” refer to biological molecules, or macromolecules composed of amino-acid residues bonding together in a chain. The definition of polypeptides used herein is intended to encompass proteins (generally higher molecular weight) composed of one or more long chains of amino acid residues and small peptides (generally lower molecular weight) of a few amino acids. In other embodiments, a single amino acid, although not technically a polypeptide, is also considered within the scope of the invention.

### DETAILED DESCRIPTION OF THE INVENTION

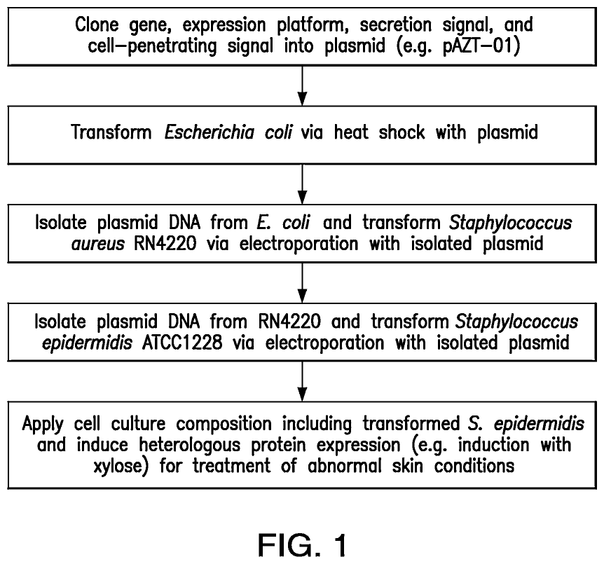

The present invention provides skin-colonizing bacteria that are genetically altered to express recombinant therapeutic polypeptides for the treatment or prevention of skin disease (FIG. 1). Using genetically engineered protein-producing bacteria has several advantages over the prior art method of treating skin disease. Therapeutic proteins are able to treat the underlying cause of defects leading to the skin condition. Further, bacteria are able to self-replicate while retaining the inserted gene to continuously produce the therapeutic protein.

The present invention provides skin-colonizing bacteria, such as for example, Staphylococcus epidermidis, that are genetically altered to express human filaggrin. Using genetically engineered filaggrin-producing bacteria has several advantages over using filaggrin supplementation. First, bacteria are able to self-replicate while retaining the inserted filaggrin gene. Second, S. epidermidis is normally present on the skin and has been shown to inhibit growth of Staphylococcus aureus, a bacterial species of the same genre that dominates the skin flora in AD flares.

### 1. Bacterial Strains

### Cell Culture Compositions

A wide range of bacteria are suitable for use in the present invention. Examples include, but are not limited to, non-pathogenic and commensal bacteria. Bacteria suitable for use in the present invention include, but are not limited to, Bifidobacterium, Brevibacterium, Propionibacterium, Lactococcus, Streptococcus, Staphylococcus (e.g., S. epidermidis), Lactobacillus (e.g., L. acidophilus), Pediococcus, Leuconostoc, or Oenococcus.

Certain embodiments involve the use of bacterium Staphylococcus epidermidis. The strain of S. epidermidis to be used is incapable of producing biofilms. An example of this is S. epidermidis strain ATCC12228.

However, other related or similar species found on the skin can also be used.

### 2. Filaggrin and Filaggrin Gene

A useful gene selected in an embodiment for this invention is a mammalian gene encoding filaggrin (FLG). Filaggrin is a protein produced by differentiating keratinocytes and functions to aggregate keratin filaments into a cytoskeleton that, in combination with other components, comprises the cornified cell envelope. FLG is a large gene located on chromosome 1q21 and produces profilaggrin, an insoluble polyprotein that is proteolyzed to release functional filaggrin monomers (Armengot-Carbo et al. 2014). The gene for this invention can be from any mammal. Non-limiting examples include mouse, rat, rabbit, goat, sheep, horse, cow, dog, primate, or human gene sequence. In preferred embodiments, the gene sequence is a human gene sequence. Non-limiting examples of filaggrin proteins are set forth in Table 1 and the sequences are further described in the corresponding Sequence IDs.

TABLE 1
Examples of filaggrin protein sequences

| Sequence | GenBank Accession No. | SEQ ID NO. |
|---|---|---|
| Filaggrin, Homo sapiens | NM_002016 | 1 |
| Filaggrin, Homo sapiens | NP_002007 | 2 |
| Filaggrin, Homo sapiens | AAA52454 | 3 |
| Filaggrin, Homo sapiens | CAI19595 | 4 |
| Filaggrin, Homo sapiens | P20930 | 5 |
| Filaggrin, Mus musculus | AAM23016 | 6 |
| Filaggrin, Mus musculus | AAA75559 | 7 |
| Filaggrin, Mus musculus | AAA37626 | 8 |
| Filaggrin, Mus musculus | AAA37625 | 9 |
| Filaggrin, Mus musculus | XP_485270 | 10 |
| Filaggrin, Mus musculus | P11088 | 11 |
| Filaggrin, Mus musculus | EDL00668.1 | 12 |
| Filaggrin, Rattus norvegicus | EDL87862 | 13 |
| Filaggrin, Pan troglodytes | XP_001134714 | 14 |
| Filaggrin, Pan troglodytes | XP_513808 | 15 |
| Filaggrin, Macaca mulatta | XP_001101725.1 | 16 |
| Filaggrin, Macaca mulatta | XP_001109011.1 | 17 |

### 3. Secretion Signals

Secretion signals or export signals are peptides on a protein that indicate the protein is destined for the secretory pathway and therefore secreted from the cell. Any secretion signal that facilitates exit of a protein such as a filaggrin molecule out of the bacterial cell is contemplated as a secretion signal. Non-limiting examples of secretion signals are set forth in Table 2.

TABLE 2
Examples of secretion signals

| Sequence | SEQ ID NO. |
|---|---|
| MKKLAFAITAASGAAAVLSHHDAEA | 18 |
| WLDNRAFSKKFVPVVMATSVALFFLNLAFA | 19 |
| MAKKFNYKLPSMVALTLFGTAFTAHQANA | 20 |
| MKKRFLSICTMTIAALATTTMVNTSYA | 21 |
| NLKKQSKLILIFICIFTFFIMIIQSQFLMG | 22 |
| MKIFKLTSLTLAALTLAFPFSHVAQA | 23 |
| MKKTVIASTLAVSLGIAGYGLSGHEAHA | 24 |
| MKKNKFLVYLLSTALITPTFATQTAFA | 25 |
| MKTRQNKYSIRKFSVGASSILIAALLFMGGGSAQA | 26 |
| MKNNNETRRFSIRKYTVGVVSIITGITIFVSGQHAQA | 27 |
| MKKKLSYMITIMLAFTLSLALGLFFNSAHA | 28 |

### 4. Cell Penetrating Peptides

A cell penetrating peptide can be used to mediate delivery of cargo in vivo without using any receptors and without causing any significant membrane damage. Cell penetrating peptides that facilitate entry into the skin keratinocytes are contemplated as a cell penetrating peptides of the present invention. Non-limiting examples are set forth in Table 3.

TABLE 3
Examples of cell penetrating peptides

| Sequence | SEQ ID NO. |
|---|---|
| GRKKRRQRRRPPQ | 29 |
| GWTLNSAGYLLGKINLKALAALAKKIL | 30 |
| KLALKLALKALKAALKLA | 31 |
| WEAKLAKALAKALAKHLAKALAKALKACEA | 32 |
| KETWWETWWTEWSQPKKKRKV | 33 |
| RRRRRRRRR | 34 |
| LGTYTQDFNKFHTFPQTAIGVGAP | 35 |

References for Seq. IDs 29-35 of Table 3: Seq ID 29. Jeong JG et al. (2011). Seq ID 30.Ostenson CG et al. (1997). Seq ID 31.Ochike J et al. (1998). Seq ID 32. Wyman TB, et al. (1997). Seq IDs 33.-35. J. Ma et al. (2014).

### 5. Genetic Construct

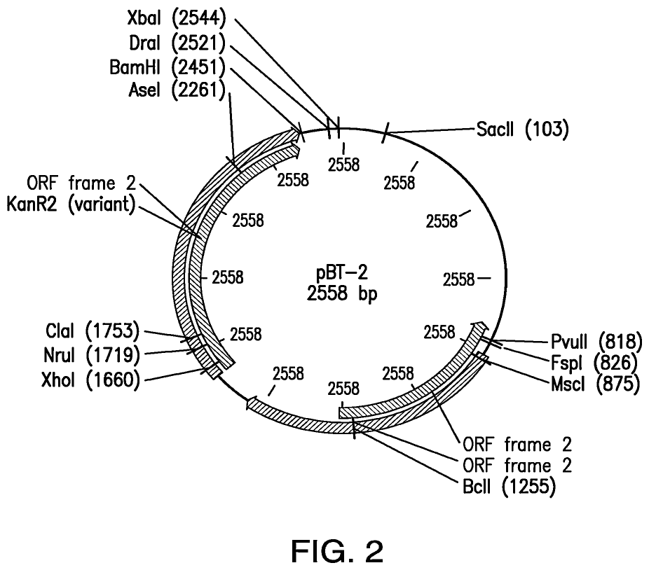

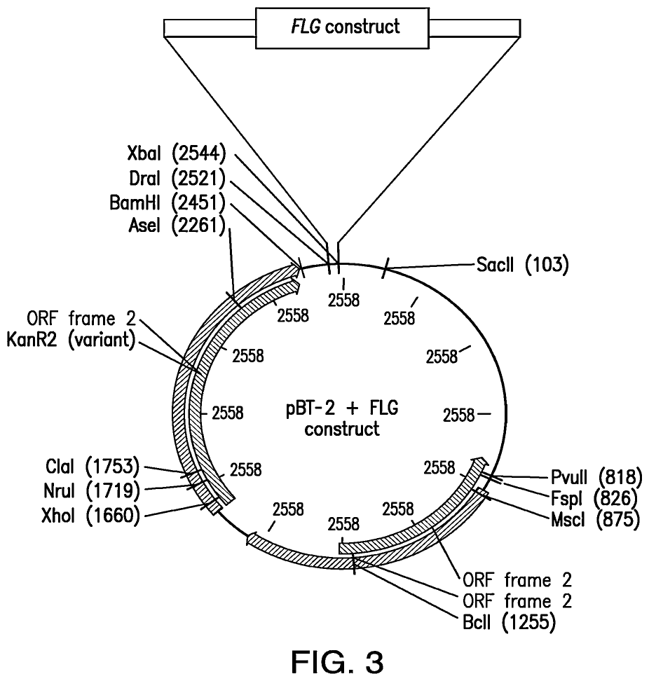

The invention utilizes standard molecular biology techniques, e.g., those described in (Sambrook et al. 2001). An example of the genetic construct used for this invention is based on a plasmid vector pBT-2 (FIG. 2), an allelic exchange shuttle vector between E. coli and Staphylococcal species (Nakanishi, Oshida et al. 1986). The plasmid of FIG. 3 is constructed by inserting cDNA of a gene encoding a therapeutic protein into a restriction site of the MCS of pBT-2. The insert contains a coding sequence driven by a promoter. Such a promoter can be either constitutive or inducible. Examples of inducible promoters include those that are activated by chemical compounds such as alcohols, sugars, metals, or tetracycline, or by physical factors such as light or high temperatures.

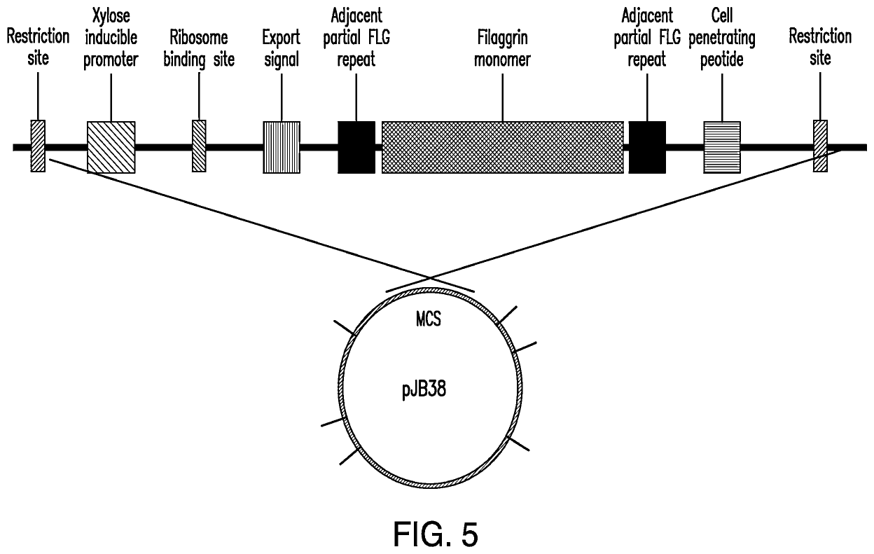

The genetic construct can be based on a plasmid vector pJB38, an allelic exchange shuttle vector between E. coli and Staphylococcal species. See (Cheung AL, et al. 2004). The mRNA sequence of human FLG has a Genebank accession No. NM_002016, and is reproduced above as SEQ. ID NO. 1. Plasmid pAZT-01 (FIG. 5) can be constructed by inserting part of the FLG cDNA into a restriction site of pJB38. The insert contains a coding sequence driven by a constitutive xylose promoter. The construct also encodes a secretion signal and a cell penetrating peptide, thus resulting in a filaggrin fusion protein.

### 6. Uses of Recombinant Bacterial Strain

It will be understood that the disorder to be treated can be any disorder associated with skin. In preferred embodiments the disorder is selected from the group comprising psoriasis, acne, atopic dermatitis, allergic contact dermatitis, epidermolytic hyperkeratosis, seborrheic dermatitis, eczema, dry skin, allergy, rashes, UV-irritated skin, detergent irritated skin (including irritation caused by enzymes and compounds used in washing detergents and sodium lauryl sulfate), thinning skin (e.g. skin from the elderly and children), bullous pemphigoid, pemphigus vulgaris, impetigo, vitiligio, baldness, and hirsutism.

Examples of proteins that can be administered according to the invention are mostly eukaryotic proteins. These can include but are not limited to single amino acids, small peptides, and large proteins. More particularly, genes encoding proteins that are useful in the invention as recombinant therapeutic proteins include, but are not limited to, the following genes. Members of the interleukin family of genes, including but not limited to IL-2, IL-3, IL-4, IL-5, IL-6, IL-7, IL-8, IL-9, IL-10, IL-11, IL-12, IL-13, IL-14 and IL-15 and genes encoding receptor antagonists thereof. Genes which encode hematopoietic growth factors, including but not limited to, erythropoietin, granulocyte colony stimulating factor, granulocyte macrophage colony stimulating factor, macrophage colony stimulating factor, stem cell factor, leukemia inhibitory factor and thrombopoietin are also contemplated in the invention. Genes encoding neurotropic factors are also contemplated, including but not limited to, nerve growth factor, brain derived neurotropic factor and ciliary neurotropic factor. In addition, genes which encode interferons, including but not limited to IFN-alpha, IFN-beta and IFN-gamma are included. Further contemplated in the invention are genes encoding chemokines such as the C-C family and the C-X-C family of cytokines, genes encoding hormones, such as proinsulin and growth hormone, and genes encoding thrombolytic enzymes, including tissue plasminogen activator, streptokinase, urokinase or other enzymes such as trypsin inhibitor. The invention further includes genes which encode tissue repair factors, growth and regulatory factors such as, but not limited to, oncostatine M, platelet-derived growth factors, fibroblast growth factors, epidermal growth factor, hepatocyte growth factor, bone morphogenic proteins, insulin-like growth factors, calcitonin and transforming growth factor alpha and beta. Further contemplated genes include genes encoding structural proteins such as filaggrin, actin, collagen, fibrillin, elastin, or scleroprotein.

### Formulations

It will be further apparent that the formulation for use according to the present invention can comprise any pharmaceutically effective amount of the recombinant bacteria to produce a therapeutically effective amount of the desired polypeptide, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0% or more by weight of recombinant bacteria, the upper limit of which is about 90.0% by weight of recombinant bacteria.

In an alternative embodiment the formulation for use according to the present invention can comprise, for example, at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to about 5%, or more by weight of recombinant bacteria.

The topical formulation for use in the present invention can be in any form suitable for application to the body surface, such as a cream, lotion, sprays, solution, gel, ointment, paste, plaster, paint, bioadhesive, suspensions, emulsions, or the like, and/or can be prepared so as to contain liposomes, micelles, and/or microspheres. Such a formulation can be used in combination with an occlusive overlayer so that moisture evaporating from the body surface is maintained within the formulation upon application to the body surface and thereafter. The formulation can include a living cell culture composition and can comprise at least one engineered bacterial strain that produces a recombinant polypeptide. This engineered living cell culture composition can deliver the polypeptide directly to the skin for treating or preventing abnormal skin conditions.

Topical formulations include those in which any other active ingredient(s) is (are) dissolved or dispersed in a dermatological vehicle known in the art (e.g. aqueous or non-aqueous gels, ointments, water-in-oil or oil-in-water emulsions). Constituents of such vehicles can comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). Depending, inter alia, upon the nature of the formulation as well as its intended use and site of application, the dermatological vehicle employed can contain one or more components (for example, when the formulation is an aqueous gel, components in addition to water) selected from the following list: a solubilizing agent or solvent (e.g. a β-cyclodextrin, such as hydroxypropyl β-cyclodextrin, or an alcohol or polyol such as ethanol, propylene glycol or glycerol); a thickening agent (e.g. hydroxyethylcellulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

A pharmaceutically acceptable carrier can also be incorporated in the formulation of the present invention and can be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers. The term “pharmaceutically acceptable” or “pharmaceutically acceptable carrier” is used herein to refer to a compound or composition that can be incorporated into a pharmaceutical formulation without causing undesirable biological effects or unwanted interaction with other components of the formulation. “Carriers” or “vehicles” as used herein refer to carrier materials suitable for incorporation in a topically applied composition. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue.

A film former, when it dries, forms a protective film over the site of application. The film inhibits removal of the active ingredient and keeps it in contact with the site being treated. An example of a film former that is suitable for use in this invention is Flexible Collodion, USP. As described in Remington: The Science and Practice of Pharmacy, 19th Ed. (Easton, PA: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoterie surfactant.

Lotions are preparations to be applied to the skin surface without friction, and are typically liquid or semiliquid preparations in which particles, including the active agent, are present in a water or alcohol base. Lotions are usually suspensions of solids, and preferably, comprise a liquid oily emulsion of the oil-in-water type. Lotions are preferred formulations herein for treating large body areas, because of the ease of applying amore fluid composition. It is generally necessary that the insoluble matter in a lotion be finely divided.

Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution can contain other pharmaceutically or cosmetically acceptable chemicals to buffer, stabilize or preserve the solute. Common examples of solvents used in preparing solutions are ethanol, water, propylene glycol or any other acceptable vehicles. As is of course well known, gels are semisolid, suspension-type systems. Single-phase gels contain organic macromolecules distributed substantially uniformly throughout the carrier liquid, which is typically aqueous, but also, preferably, contain an alcohol, and, optionally, an oil. Preferred “organic macromolecules,” i.e., gelling agents, are cross-linked acrylic acid polymers such as the “carbomer” family of polymers, e.g., carboxypolyalkylenes that can be obtained commercially under the Carbopol trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, PA: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little orno water and include, for example, hydroxystearin sulfate, anhydrous lanolin, and hydrophilic petrolatum.

Emulsion ointment bases are either water-in-oil (W/O) emulsions or oil-in-water (O/W) emulsions, and include, for example, acetyl alcohol, glyceryl monostearate, lanolin, and stearic acid. Preferred water-soluble ointment bases are prepared from polyethylene glycols of varying molecular weight; see Remington: The Science and Practice of Pharmacy for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Enhancers are those lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter 8 of plasticizing enhancers is in the range of about 2.5 to about 10, preferably in the range of about 5 to about 10. Preferred lipophilic enhancers are fatty esters, fatty alcohols, and fatty ethers. Examples of specific and most preferred fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995) (incorporated herein by reference).

Various other additives can be included in the compositions of the present invention in addition to those identified above. These include, but are not limited to, antioxidants, astringents, perfumes, preservatives, emollients, pigments, dyes, humectants, propellants, and sunscreen agents, as well as other classes of materials whose presence can be pharmaceutically or otherwise desirable. Typical examples of optional additives for inclusion in the formulations of the invention are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (vitamin C), a-tocopherol (Vitamin E), β-tocopherol, γ-tocopherol, δ-tocopherol, ε-tocopherol, ζι-tocopherol, Z^{Λ}-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the Carbopol trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-trichloro-2-hydroxy diphenyl ether (triclosan) and benzoic acid; anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants such as ascorbic acid (Vitamin C), a-tocopherol (Vitamin E), β-tocopherol, γ-tocopherol, δ-tocopherol, ε-tocopherol, ζι-tocopherol, ζ₂-tocopherol, η-tocopherol, and retinol (Vitamin A), and/or pharmaceutically acceptable salts, esters, amides, or other derivatives thereof. A preferred tocopherol compound is a-tocopherol. Additional agents include those that are capable of improving oxygen supply in skin tissue, as described, for example, in Gross, et al. WO 94/00098 and Gross, et al, WO 94/00109, both assigned to Lancaster Group AG (incorporated herein by reference). Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycinnamate, octyl salicylate, oxybenzone, padimate O, phenylbenzimidazole sulfonic acid, sulisobenzone, titanium dioxide, trolamine salicylate, zinc oxide, ensulizole, meradimate, octinoxate, octisalate, and octocrylene. See Title 21. Chapter 1. Subchapter D. Part 352. “Sunscreen drug products for over-the-counter human use” incorporated herein in its entirety.

Other embodiments can include a variety of non-carcinogenic, non-irritating healing materials that facilitate treatment with the formulations of the invention. Such healing materials can include nutrients, minerals, vitamins, electrolytes, enzymes, herbs, plant extracts, glandular or animal extracts, or safe therapeutic agents that can be added to the formulation to facilitate the healing of dermal disorders.

The amounts of these various additives are those conventionally used in the cosmetics field, and range, for example, from about 0.01% to about 20% of the total weight of the topical formulation.

The formulations of the invention can also include conventional additives such as opacifiers, fragrance, colorant, stabilizers, surfactants, and the like. In certain embodiments, other agents can also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof. In other embodiments, other agents can also be added, such as repressors and inducers, i.e., to inhibit (i.e. glycose) or induce (i.e. xylose) the production of the polypeptide of interest. Such additives can be employed provided they are compatible with and do not interfere with the function of the formulations.

The formulations can also contain irritation-mitigating additives to minimize or eliminate the possibility of skin irritation or skin damage resulting from the chemical entity to be administered, or other components of the composition.

Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphiphilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than about 20 wt. %, more typically not more than about 5 wt. %, of the formulation.

Further suitable pharmacologically active agents that can be incorporated into the present formulations in certain embodiments and thus topically applied along with the active agent include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites.

Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycinnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The application regimen will depend on a number of factors that can readily be determined, such as the severity of the condition and its responsiveness to initial treatment, but will normally involve one or more applications per day on an ongoing basis. One of ordinary skill can readily determine the optimum amount of the formulation to be administered, administration methodologies and repetition rates. In general, it is contemplated that the formulations of the invention will be applied in the range of once or twice weekly up to once or twice daily.

As described herein, the bacteria species selected for the composition is transformed using known recombinant techniques to express a compound of interest.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1 Staphylococcus epidermidis Expressing the Element Filaggrin Using Vector pAZT-01

### A. Bacteria

Bacteria of the Staphylococcus aureus RN4220 strain were used in preparation of the vector (Kreiswirth, BN., et al. 1983). A stock solution of the strain was stored in −20° C. in 50% glycerol in brain heart fusion broth (BHI).

Bacteria of the Staphylococcus epidermidis strain ATCC12228 were used (Zhang, YQ., et al. 2003). A stock solution of the strain was stored in −20° C. in 50% glycerol in brain heart fusion broth (BHI). Bacteria were cultured in brain heart fusion broth (BHI). After 16 hours of incubation, bacteria were harvested by centrifugation and 10-fold concentrated in brain heart fusion broth (BHI) at 2×10⁹ bacteria/100 μl. A stock preparation of the bacteria was prepared by inoculating 5 mL broth with cells from the slant. The cells were grown overnight at 30° C. Then 3 mL fully grown culture was added to 1 ml 60% glycerol and stored at −80° C.

### B. Genomic Integration Into the Vector

Plasmid AZT-01 consists of shuttle vector pJB38 with a filaggrin expression insert (FIG. 5) (Cheung, AL., et al. 2004). The insert into pJB38 contains the complete operon for a filaggrin DNA sequence (Genbank accession no. NM_002016), a xylose-inducible promoter and was used in the production of the compound of interest. The pJB38-FLG vector (FIG. 5) was constructed and cloned by Bio Basic, Inc. (Markman, ON, Canada).

### C. Transformation

A vector harboring the filaggrin sequence, or any vector constructed for the purpose of therapeutic treatment of abnormal human skin was transformed into the S. epidermidis strain, according to the following protocol, including the following steps: preparation of S. aureus bacterial cells, transformation of S. aureus, isolation of plasmid DNA from S. aureus, preparation of S. epidermidis bacterial cells, transformation of S. epidermidis, growth of bacteria, and storage of transformed bacteria. Alternative intermediate strains can also be used for transformation and isolation of plasmid DNA in preparation for transformation into S. epidermidis. These strains include but are not limited to S. aureus and E. coli strains, including those deficient in methylation.

Preparation and transformation of S. aureus: Preparation of the cells: S. aureus RN4220 cells were made electrocompetent by growing 50 ml culture overnight in BHI medium at 37° C., then inoculating 100 ml fresh BHI medium with 10 ml of overnight culture. When OD₆₀₀ reached 0.2-0.3, cells were pelleted, and resuspended with 1× volume of 4° C. 10% sucrose. This process was repeated 3×, then the cells were resuspended with 0.1× volume of 4° C. 10% sucrose, pelleted, and resuspended with 1 ml of 10% sucrose.

For transformation of RN4220, 200-500 mg of pAZT-01 was mixed with electrocompetent cells previously prepared and transformed using electroporation at room temperature at 2.5 kV using the MicroPulser Electroporator (Bio-Rad, Hercules, CA). Transformed cells were plated at 28° C. overnight on selective BHI medium, grown overnight in selective BHI medium and then used to isolate DNA.

Preparation and transformation of S. epidermidis: Electrocompetent S. epidermidis ATCC12228 were made using the following methods. First, 50 ml overnight culture of ATCC12228 from a −80° C. stock were grown at 37° C. in B2 medium (1.0% tryptone, 2.5% yeast extract, 0.5% glucose, 2.5% NaCl, 0.1% K₂PO₄, pH to 7.5). 10 ml of overnight culture were diluted into fresh pre-warmed B2 media and shaken until OD₆₀₀ reached 0.5-0.6, and were then pelleted from 10 min at 4° C. Next, cells were washed with 1, ½, 1/20, and 1/50 volumes of cold 10% glycerol, pelleting at 4° C. between washes. The final pellet was resuspended in 700 ul of cold 10% glycerol.

Electrocompetent ATCC12228 were transformed with pAZT-01, isolated from S. aureus, using electroporation at 2.5 kV, 25 uF. 100 Ω. (normal reading is 4.5-5 msec using the Micropulser Electroporator (Bio-Rad, Hercules, CA)). Cells were then plated at 28° C. on selective BHI medium.

Transformation of the bacteria can also be performed via alternative methods of transformation including but not limited to alternative intermediate strains, bacteriophage transduction, and heat shock.

### D. Induction of Protein Expression

Filaggrin expression was induced in ATCC12228 transformed with pAZT-01 in the following manner. First, 5 ml of an overnight culture at 28° C. of transformed ATCC12228 in selective BHI medium was taken to inoculate 100 ml of fresh BHI with antibiotic. The cells were grown until OD₆₀₀ reached 0.5-0.65, then the cells were induced using a final concentration of 1.5% xylose for 4.5 hours.

### E. Analysis of Protein Expression and Secretion

For analysis cells were fractionated and analyzed via SDS-PAGE electrophoresis and western blotting. Bacterial cells from un-induced samples and induced samples were pelleted and lysed with CelLytic B Cell Lysis Reagent (Sigma-Aldrich, St. Louis, MO). The supernatant from the induced sample was also collected and concentrated. Samples were resuspended in a reduced sample buffer and then electrophoresed on a 4-15% Tris gel with Tris-HCL running buffer. Following electrophoresis, the gel was transferred to a PVDF membrane, and sequentially probed with a primary goat monoclonal antibody against filaggrin (sc-25897, Santa Cruz Biotechnology, Inc). A horseradish peroxidase-conjugated donkey anti-goal antibody (sc-2020) was then probed and the secondary antibodies were detected through autoradiography (Syngene GeneGnome Bio Imaging System) using enhanced chemiluminescence substrate (SuperSignal West Pico, Thermo Scientific).

Analysis of the supernatant and cell lysate demonstrates the successful expression and secretion of the therapeutic polypeptide upon transformation with plasmid containing protein of interest, followed by induction of protein expression.

Detection of protein expression and secretion is also possible using alternative methods and the current example should not be construed as a limitation to the present invention.

### F. Treatment of Human Subjects With Foregoing Composition

Approximately 1×10⁹ colony forming units (CFU) of S. epidermidis containing pAZT-01 can be added to a pharmaceutically acceptable carrier. The foregoing composition is useful for treating or preventing abnormal skin conditions in a subject in need thereof, e.g., a human subject having an abnormal skin condition such as eczema. The composition can be applied at least once per day, up to for example about 3 to 4 times per day, or as needed or prescribed. The composition can be used for as long as needed to ensure treatment of the condition or to continue to prevent the condition. The duration of treatment can vary from about 1 day up to about 10 to 14 days or longer. In certain instances, long term or chronic treatment can be administered.

### Example 2 Staphylococcus epidermidis Colonizing Epidermal Layer and Expressing GFP

### A. Bacteria

Bacteria of the Escherichia coli strains DH5α and DC10B were used in plasmid construction and preparation (Sambrook, J., et al. 1989; Monk, I., et al. 2012). Bacteria of the Staphylococcus epidermidis ATCC12228 strain were used for heterologous expression of GFP in the murine colonization and expression studies. A stock solution of the strain was stored in −20° C. in 50% glycerol in brain heart fusion broth (BHI). Bacteria were cultured in brain heart fusion broth (BHI). After 16 hours of incubation, bacteria were harvested by centrifugation and 10-fold concentrated in BHI at 2×10⁹ bacteria/100 μl. A stock preparation of the bacteria was prepared by inoculating 5 mL broth with cells from the slant. The cells were grown overnight at 30° C. Then 3 mL fully grown culture was added to 1 ml 60% glycerol and stored at −80° C.

### B. Vector

Plasmid pCM11 consists of shuttle vector with a sGFP expression insert (Lauderdale et al. 2010).

### C. Transformation

A vector harboring the GFP sequence (e.g. pCM11 (Lauderdale et al. 2010)), or any vector constructed for the purpose of heterologous expression was transformed into the S. epidermidis strain ATCC12228, according to the following protocol, including the following steps: transformation into DH5α, isolation of plasmid DNA from DH5α, transformation of DC10B, isolation of plasmid DNA from DC10B, preparation and transformation of S. epidermidis, growth and application of transformed S. epidermidis.

E. coli DH5α was transformed with pCM11 using the heat shock method and plated on selective LB (Luria Broth) overnight at 37° C. Plasmid DNA was purified from 37° C. overnight cultures with selective LB media from DH5α transformants using Zymo Research Plasmid Miniprep Kit (Irvine, CA). Next, 200-500 ng pCM11 DNA was used to transform DC10B using electroporation at room temperature at 2.5 kV using the MicroPulser Electroporator (Bio-Rad, Hercules, CA). The transformed cells were plated overnight at 37° C. and then DNA was purified using the said method.

Next, electrocompetent S. epidermidis ATCC12228 were made using the following methods. First, 50 ml overnight culture of ATCC12228 were grown at 37° C. in B2 medium (1.0% tryptone, 2.5% yeast extract, 0.5% glucose, 2.5% NaCl, 0.1% K₂PO₄, pH to 7.5). 10 ml of overnight culture were diluted into fresh pre-warmed B2 media and shaken until OD₆₀₀ reached 0.5-0.6, and were then pelleted from 10 min at 4° C. Next, cells were washed with 1, ½, 1/20, and 1/50 volumes of cold 10% glycerol, pelleting at 4° C. between washes. The final pellet was resuspended in 700 ul of cold 10% glycerol.

Electrocompetent ATCC12228 were transformed with pCM11, isolated from DC10B, using electroporation at 2.5 kV using the Micropulser Electroporator (Bio-Rad, Hercules, CA). Cells were then plated at 37° C. on selective BHI medium.

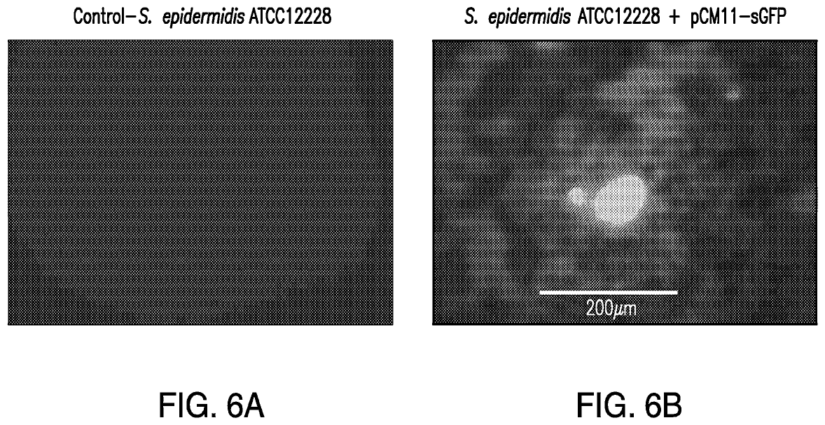

Transformation and expression of the GFP insert was confirmed using microscopy as seen by the fluorescence of transformed S. epidermidis ATCC12228 in FIG. 6.

D. Murine Studies With ATCC12228 Transformed With pCM11

In order to visualize colonization of the ATCC12228 onto mouse skin, mice were treated with GFP-expressing ATCC12228 (ATCC12228 transformed with pCM11). A small cohort of mice (n=5) were clipped to remove hair and treated on dorsal skin with S. epidermidis ATCC12228 expressing GFP in pCM11. Approximately 1×10⁹ CFUs of S. epidermidis were applied with a cotton wool tipped stick. After initiation of treatment, the mice were monitored for signs of S. epidermidis colonization.

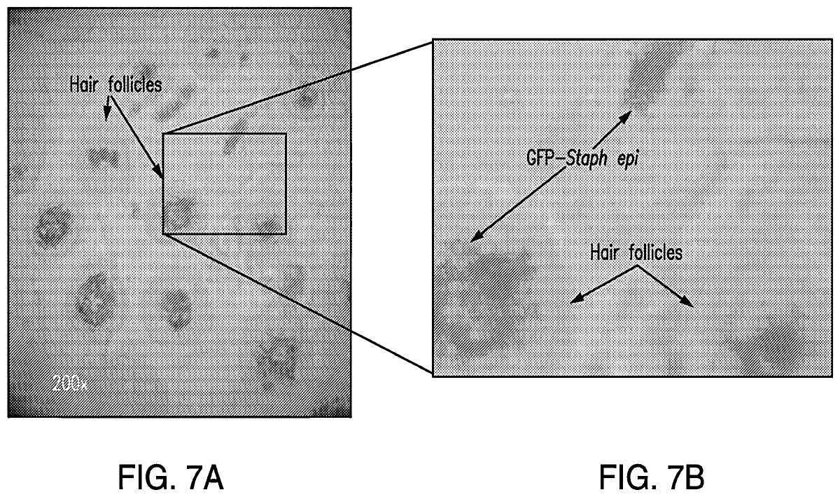

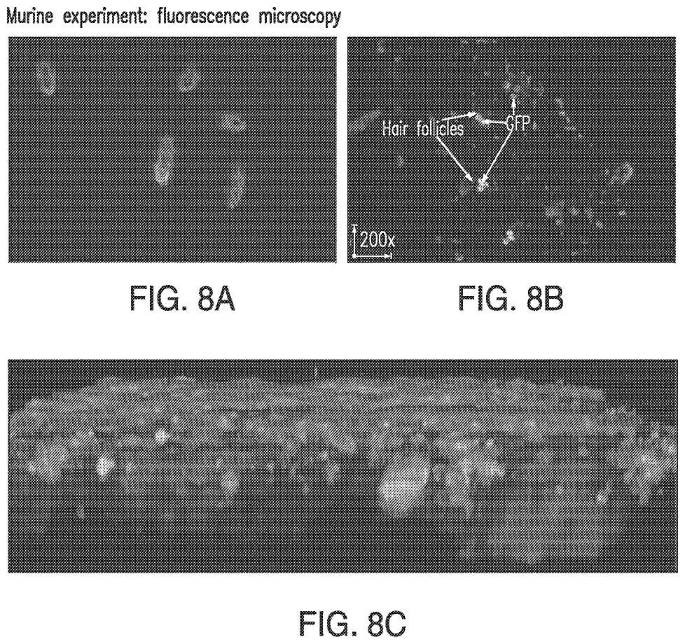

Sections of skin were taken from skin post mortem for imaging studies. Sections were imaged using fluorescent microscopy (FIG. 7) including staining with DAPI and an anti-GFP monoclonal antibody (FIG. 8). Additional images were visualized using two-photon microscopy (FIG. 8).

FIG. 7 depicts S. epidermidis ATCC12228 transformed with pCM11 containing the GFP insert, which was in turn applied on the dorsal skin of mice for three days. Sections of the skin were then taken post-mortem for light microscopy (FIG. 7A & 7B). Sections shown here were stained using Malachite Green stain and visualized at 200× (FIG. 7A), and further expansion in FIG. 7B. This figure demonstrates the colonization of S. epidermidis on the mouse skin after three days.

FIG. 8 depicts S. epidermidis ATCC12228 transformed with pCM11 containing the GFP insert, which was in turn applied on the dorsal skin of mice for three days. Sections of the skin were then taken post-mortem for light microscopy (FIG. 8A & 8B). FIG. 8A shows fluorescence of GFP indicating not only that S. epidermidis colonizes the skin after three days, but also that expression of GFP is maintained at that time. FIG. 8A shows fluorescence from GFP while FIG. 8B shows a section of skin stained with both DAPI (4′,6-diamidino-2-phenylindole) and an anti-GFP monoclonal antibody. FIG. 8C demonstrates a section of the skin visualized using two-photon microscopy. This image demonstrates the extent of S. epidermidis colonization and GFP expression, as indicated by the fluorescence.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### Incorporation by Reference

The entire disclosure of each of the patent documents, including patent application documents, scientific articles, governmental reports, websites, and other references referred to herein is incorporated by reference herein in its entirety for all purposes. In case of a conflict in terminology, the present specification controls.

All sequence listings, or Seq. ID. Numbers, disclosed herein are incorporated herein in their entirety.

### Equivalents

The invention can be embodied in other specific forms without departing from the spirit or essential characteristics thereof. The foregoing embodiments are to be considered in all respects illustrative rather than limiting on the invention described herein. In the various embodiments of the methods and compositions of the present invention, where the term comprises is used with respect to the recited steps or components, it is also contemplated that the methods and compositions consist essentially of, or consist of, the recited steps or components. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

### References

The following references, to the extent that they provide exemplary procedural or other details supplementary to those set forth herein, are specifically incorporated herein by reference.
Armengot-Carbo, M. et al. (2014) “The role of filaggrin in the skin barrier and disease development.” Actas Dermosifiliogr Mar; 106 (2):86-95.
Brachkova, M. I., P. Marques, J. Rocha, B. Sepodes, M. A. Duarte and J. F. Pinto (2011). “Alginate films containing Lactobacillus plantarum as wound dressing for prevention of burn infection.” J Hosp Infect 79(4): 375-377.
Brown, SJ., & McLean, WH. (2012) J. Invest. Dermatol. 132, 751-62
Chen, YE., & Tsao, H. (2013) J. Am. Acad. Dermatol. 69, 143-155
Cheung AL, et al. (2004) “Regulation of virulence determinants in vitro and in vivo in Staphylococcus aureus.” FEMS Immunological Medical Microbiology 40(1):1-9
“DNA Recombination.” Methods in Molecular Biology 745(XIV): 1-565.
Gross, et al., WO 94/00098 assigned to Lancaster Group AG
Gross, et al., WO 94/00109 assigned to Lancaster Group AG
Gueniche, A., P. Bastien, J. M. Ovigne, M. Kermici, G. Courchay, V. Chevalier, L. Breton and I. Castiel-Higounenc (2010). “Bifidobacterium longum lysate, a new ingredient for reactive skin.” Exp Dermatol 19(8): 1-8.
Jeong JG et al. (2011). A Tat-grafted anti-nucleic acid antibody acquires nuclear-localization property and a preference for TAR RNA. Biochem Biophys Res Commun. Mar 18;406(3):403-7.
Kreiswirth, BN., et al. (1983). The toxic shock syndrome exotoxin structural gene is not detectably transmitted by a prophage. Nature 305:709-712.
Lauderdale, et al. (2010). Biofilm dispersal of community-associated methicillin-resistant Staphylococcus aureus on orthopedic implant material. J. Orthop. Research. 28:55-61
Lee, SH., Jeong, SK. and Ahn, SK. (2006). “An update of the defensive barrier function of skin.” Yonsei Med J 47(3): 293-306.
Lin, YT., Wang, CT., and Chiang, BL. (2007). “Role of bacterial pathogens in atopic dermatitis.” Clin Rev Allergy Immunol 33(3): 167-177.
Ma, J., et al. (2014) Cell-penetrating peptides mediated protein cross-membrane delivery and its use in bacterial vector vaccine. Fish & Shellfish Immunology 39 8-16
McAleer, MA., & Irvine, AD. (2013) J. Allergy Clin. Immunol. 131, 280-91.
Monk, I., et al. (2012) Direct transformation to manipulate genetically Staphylococcus aureus and Staphylococcus epidermidis. mBio.
Muizzuddin, N., Maher, W., Sullivan, M., Schnittger, S., and Mammone, T. (2012). “Physiological effect of a probiotic on skin.” J Cosmet Sci 63(6): 385-395.
Nakanishi, N., T. Oshida, S. Yano, K. Takeda, T. Yamaguchi and Y. Ito (1986). “Construction and characterization of new cloning vectors derived from Streptomyces griseobrunneus plasmid pBT1 and containing amikacin and sulfomycin resistance genes.” Plasmid 15(3): 217-229.
Nakatsuji, T. and R. L. Gallo (2014). “Dermatological therapy by topical application of non-pathogenic bacteria.” J Invest Dermatol 134(1): 11-14.
Oehike J et al. (1998).Cellular uptake of an alpha-helical amphipathic model peptide with the potential to deliver polar compounds into the cell interior non-endocytically. Biochim Biophys Acta. Nov 11;1414(1-2):127-39.
Ostenson CG et al. (1997).Galparan: a powerful insulin-releasing chimeric peptide acting at a novel site. Endocrinology. Aug;138(8):3308-13.
Otsuka, A., et al. (2014) J. Allergy Clin. Immunol. 133, 139-46.e1-10 (2014).
Peral, M. C., M. A. Martinez and J. C. Valdez (2009). “Bacteriotherapy with Lactobacillus plantarum in burns.” Int Wound J 6(1): 73-81.
Peral, M. C., M. M. Rachid, N. M. Gobbato, M. A. Huaman Martinez and J. C. Valdez (2010). “Interleukin-8 production by polymorphonuclear leukocytes from patients with chronic infected leg ulcers treated with Lactobacillus plantarum.” Clin Microbiol Infect 16(3): 281-286
Powers, ME., et al. (2011). J Bacteriol., 193:340-348
Proksch, E., J. M. Brandner and J. M. Jensen (2008). “The skin: an indispensable barrier.” Exp Dermatol 17(12): 1063-1072
Remington: The Science and Practice of Pharmacy, 19^{th} edition. Easton, PA: Mack Publishing Co., 1995
Sambrook J, et al. (1989). Molecular Cloning: A Laboratory Manual.Cold Spring Harbor Laboratory Press, New York.
Sambrook, JF., and Russell, DW., ed. (2001). Molecular Cloning: A Laboratory Manual, 3rd ed., Vols 1, 2 and 3. Cold Spring Harbor Laboratory Press
Simonen, M. and I. Palva (1993). “Protein secretion in Bacillus species.” Microbiol Rev 25 57(1): 109-137
Smith, EW., & Maibach, HI., (1995) Percutaneous Penetration Enhancers, CRC Press ISBN 9780849321528
Stout, TE., et al. (2014) J Invest Dermatol. 134, 423-9

The Science and Practice of Pharmacy (1995), 19th Ed. Easton, PA: Mack Publishing Co.
Volz, T., Y. Skabytska, E. Guenova, K. M. Chen, J. S. Frick, C. J. Kirschning, S. Kaesler,
   M. Rocken and T. Biedermann (2014). “Nonpathogenic bacteria alleviating atopic dermatitis inflammation induce IL-10-producing dendritic cells and regulatory Tr1 cells.” J Invest Dermatol 134(1): 96-104
Webb, TR., & Hsu, CPS. U.S. Pat. No. 4,659,774 assigned to American Hoechst Corporation
Wyman TB, et al. (1997) Design, synthesis, and characterization of a cationic peptide that binds to nucleic acids and permeabilizes bilayers. Biochemistry. March 11;36(10):3008-17
Zhang, YQ., et al. (2003). Genome-based analysis of virulence genes in a non-biofilm-forming Staphylococcus epidermidis strain (ATCC12228). Molecular Microbiology 49(6), 1577-1593

What is claimed is:

1. A method of treating or preventing an abnormal skin condition in a human in need thereof, the method comprising administering to said human a cell culture composition comprising a bacterial strain selected from Bifidobacterium, Brevibacterium, Propionibacterium, Lactococcus, Streptococcus, Staphylococcus, Lactobacillus, Enterococcus, Pediococcus, Leuconostoc, or Oenococcus, and mixtures thereof, wherein the cell culture composition comprises at least one engineered bacterial strain that produces a therapeutically effective amount of a recombinant polypeptide for treating or preventing the abnormal skin condition.

2. A method according to claim 1 wherein said composition comprises a bacterial strain containing human target DNA recombined with bacterial DNA.

3. A method according to claim 1 wherein the composition comprises an engineered strain of Staphylococcus epidermidis.

4. A method according to claim 1 wherein the cell culture composition is a living cell culture composition.

5. A method according to claim 3, wherein the skin condition is selected from psoriasis, acne, atopic dermatitis, allergic contact dermatitis, epidermolytic hyperkeratosis, eczema, seborrheic dermatitis, dry skin, allergy, rashes, UV-irritated skin, detergent irritated skin, thinning skin, bullous pemphigoid, pemphigus vulgaris, impetigo, vitiligio, baldness, and hirsutism.

6. A method according to claim 5, wherein the skin condition is eczema.

7. A method according to claim 6, wherein said eczema is a mild eczema.

8. A method according to claim 6, wherein said eczema is a moderate eczema.

9. A method according to claim 6, wherein said eczema is a severe eczema.

10. A method according to claim 6, wherein said eczema is hand eczema.

11. A method of treating or preventing an abnormal skin condition in a human in need thereof, the method comprising administering to said human a cell culture composition comprising a bacterial strain selected from Bifidobacterium, Brevibacterium, Propionibacterium, Lactococcus, Streptococcus, Staphylococcus, Lactobacillus, Enterococcus, Pediococcus, Leuconostoc, or Oenococcus, and mixtures thereof, wherein the cell culture composition comprises at least one engineered bacterial strain that produces a therapeutically effective amount of a recombinant polypeptide comprising (a) a filaggrin amino acid sequence, (b) a secretion signal, and (c) a cell penetrating peptide, for treating or preventing the abnormal skin condition.

12. A method according to claim 11 wherein the cell culture composition comprises Staphylococcus epidermidis.

13. A method according to claim 11, wherein the cell culture composition is a living culture composition.

14. A method according to claim 11, wherein the filaggrin amino acid sequence comprises a sequence selected from SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, or SEQ ID NO 17, and combinations thereof.

15. A method according to claim 11, wherein the filaggrin amino acid sequence comprises SEQ ID NO 1.

16. A method according to claim 11, wherein the filaggrin amino acid sequence has at least about 75% homology to SEQ ID NO 1.

17. A method according to claim 11, wherein the filaggrin amino acid sequence has at least about 80% homology to SEQ ID NO 1.

18. A method according to claim 11, wherein the filaggrin amino acid sequence has at least about 85% homology to SEQ ID NO 1.

19. A method according to claim 11, wherein the filaggrin amino acid sequence has at least about 90% homology to SEQ ID NO 1.

20. A method according to claim 11, wherein the filaggrin amino acid sequence has at least about 95% homology to SEQ ID NO 1.

21. A method according to claim 11, wherein the skin condition is selected from psoriasis, acne, atopic dermatitis, allergic contact dermatitis, epidermolytic hyperkeratosis, seborrheic dermatitis, dry skin, eczema, allergy, rashes, UV-irritated skin, detergent irritated skin, thinning skin, bullous pemphigoid, pemphigus vulgaris, impetigo, vitiligio, baldness, and hirsutism.

22. A method according to claim 21, wherein the skin condition is eczema.

23. A pharmaceutical composition for treating or preventing an abnormal skin condition in a human in need thereof, comprising (a) a cell culture composition comprising a bacterial strain selected from Bifidobacterium, Brevibacterium, Propionibacterium, Lactococcus, Streptococcus, Staphylococcus, Lactobacillus, Enterococcus, Pediococcus, Leuconostoc, or Oenococcus, and mixtures thereof, wherein the cell culture composition comprises at least one engineered bacterial strain that produces a therapeutically effective amount of a recombinant polypeptide for treating or preventing the abnormal skin condition and (b) a pharmaceutically acceptable carrier.

24. A composition according to claim 23 wherein said composition comprises a bacterial strain containing human target DNA recombined with bacterial DNA.

25. A composition according to 23, wherein the composition comprises an engineered strain of Staphylococcus epidermidis.

26. A composition according to claim 23 wherein the cell culture composition is a living cell culture composition.

27. A composition according to claim 23, wherein the composition comprises 0% water to no more than about 90% water.

28. A composition according to claim 23, wherein the pharmaceutically acceptable carrier is selected from an aqueous solution, an emulsion, a cream, a lotion, a gel, or an ointment.

29. A pharmaceutical composition for treating or preventing an abnormal skin condition in a human in need thereof, comprising (I) a cell culture composition comprising a bacterial strain selected from Bifidobacterium, Brevibacterium, Propionibacterium, Lactococcus, Streptococcus, Staphylococcus, Lactobacillus, Enterococcus, Pediococcus, Leuconostoc, or Oenococcus, and mixtures thereof, wherein the cell culture composition comprises at least one engineered bacterial strain that produces a therapeutically effective amount of a recombinant polypeptide comprising (a) a filaggrin amino acid sequence, (b) a secretion signal, and (c) a cell penetrating peptide and (II) a pharmaceutically acceptable carrier, for treating or preventing the abnormal skin condition.

30. A composition according to claim 29 wherein said composition comprises a bacterial strain containing human target DNA recombined with bacterial DNA.

31. A pharmaceutical composition according to 29, wherein the cell culture composition comprises Staphylococcus epidermidis.

32. A composition according to claim 29 wherein the cell culture composition is a living cell culture composition.

33. A pharmaceutical composition according to claim 29, wherein the filaggrin amino acid sequence comprises a sequence selected from SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, or SEQ ID NO 17, and combinations thereof.

34. A pharmaceutical composition according to claim 29, wherein the filaggrin amino acid sequence comprises SEQ ID NO 1.

35. A pharmaceutical composition according to claim 29, wherein the filaggrin amino acid sequence has at least about 75% homology to SEQ ID NO 1.

36. A pharmaceutical composition according to claim 29, wherein the filaggrin amino acid sequence has at least about 80% homology to SEQ ID NO 1.

37. A pharmaceutical composition according to claim 29, wherein the filaggrin amino acid sequence has at least about 85% homology to SEQ ID NO 1.

38. A pharmaceutical composition according to claim 29, wherein the filaggrin amino acid sequence has at least about 90% homology to SEQ ID NO 1.

39. A pharmaceutical composition according to claim 29, wherein the filaggrin amino acid sequence has at least about 95% homology to SEQ ID NO 1.

40. A composition according to claim 29, wherein the composition comprises 0% water to no more than about 90% water.

41. A composition according to claim 29, wherein the pharmaceutically acceptable carrier is selected from an aqueous solution, an emulsion, a cream, a lotion, a gel, or an ointment.

42. The use of a cell culture composition for the manufacture of a medicament for treating or preventing an abnormal skin condition in a human in need thereof, said cell culture composition comprising a bacterial strain selected from Bifidobacterium, Brevibacterium, Propionibacterium, Lactococcus, Streptococcus, Staphylococcus, Lactobacillus, Enterococcus, Pediococcus, Leuconostoc, or Oenococcus, and mixtures thereof, wherein the cell culture composition comprises at least one engineered bacterial strain that produces a therapeutically effective amount of a recombinant polypeptide for treating or preventing the abnormal skin condition.

43. The use of a cell culture composition for the manufacture of a medicament for treating or preventing an abnormal skin condition in a human in need thereof, said cell culture composition comprising a bacterial strain selected from Bifidobacterium, Brevibacterium, Propionibacterium, Lactococcus, Streptococcus, Staphylococcus, Lactobacillus, Enterococcus, Pediococcus, Leuconostoc, or Oenococcus, and mixtures thereof wherein the cell culture composition comprises at least one engineered bacterial strain that produces a therapeutically effective amount of a recombinant polypeptide comprising (a) a filaggrin amino acid sequence. (b) a secretion signal. and (c) a cell penetrating peptide. for treating or preventing the abnormal skin condition.


SEQUENCE LISTING (35 sequences)

SEQ ID NO: 1 - protein, 4061 aa, Homo sapiens
MSTLLENIFAIINLFKQYSKKDKNTDTLSKKELKELLEKEFRQILKNPDDPDMVDVFMDHLDIDHNKKIDFTEFLLMVFKLAQAYYESTRKENLPISGHKHRKHSHHDKHEDNKQEENKENRKRPSSLERRNNRKGNKGRSKSPRETGGKRHESSSEKKERKGYSPTHREEEYGKNHHNSSKKEKNKTENTRLGDNRKRLSERLEEKEDNEEGVYDYENTGRMTQKWIQSGHIATYYTIQDEAYDTTDSLLEENKIYERSRSSDGKSSSQVNRSRHENTSQVPLQESRTRKRRGSRVSQDRDSEGHSEDSERHSGSASRNHHGSAWEQSRDGSRHPRSHDEDRASHGHSADSSRQSGTRHAETSSRGQTASSHEQARSSPGERHGSGHQQSADSSRHSATGRGQASSAVSDRGHRGSSGSQASDSEGHSENSDTQSVSGHGKAGLRQQSHQESTRGRSGERSGRSGSSLYQVSTHEQPDSAHGRTGTSTGGRQGSHHEQARDSSRHSASQEGQDTIRGHPGSSRGGRQGSHHEQSVNRSGHSGSHHSHTTSQGRSDASHGQSGSRSASRQTRNEEQSGDGTRHSGSRHHEASSQADSSRHSQVGQGQSSGPRTSRNQGSSVSQDSDSQGHSEDSERWSGSASRNHHGSAQEQSRDGSRHPRSHHEDRAGHGHSADSSRKSGTRHTQNSSSGQAASSHEQARSSAGERHGSRHQLQSADSSRHSGTGHGQASSAVRDSGHRGSSGSQATDSEGHSEDSDTQSVSGHGQAGHHQQSHQESARDRSGERSRRSGSFLYQVSTHKQSESSHGWTGPSTGVRQGSHHEQARDNSRHSASQDGQDTIRGHPGSSRRGRQGSHHEQSVDRSGHSGSHHSHTTSQGRSDASRGQSGSRSASRTTRNEEQSRDGSRHSGSRHHEASSHADISRHSQAGQGQSEGSRTSRRQGSSVSQDSDSEGHSEDSERWSGSASRNHRGSAQEQSRHGSRHPRSHHEDRAGHGHSADSSRQSGTPHAETSSGGQAASSHEQARSSPGERHGSRHQQSADSSRHSGIPRRQASSAVRDSGHWGSSGSQASDSEGHSEESDTQSVSGHGQDGPHQQSHQESARDWSGGRSGRSGSFIYQVSTHEQSESAHGRTRTSTGRRQGSHHEQARDSSRHSASQEGQDTIRAHPGSRRGGRQGSHHEQSVDRSGHSGSHHSHTTSQGRSDASHGQSGSRSASRQTRKDKQSGDGSRHSGSRHHEAASWADSSRHSQVGQEQSSGSRTSRHQGSSVSQDSDSERHSDDSERLSGSASRNHHGSSREQSRDGSRHPGFHQEDRASHGHSADSSRQSGTHHTESSSHGQAVSSHEQARSSPGERHGSRHQQSADSSRHSGIGHRQASSAVRDSGHRGSSGSQVTNSEGHSEDSDTQSVSAHGQAGPHQQSHKESARGQSGESSGRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQSESAHGRTGPSTGGRQRSRHEQARDSSRHSASQEGQDTIRGHPGSSRGGRQGSHYEQSVDSSGHSGSHHSHTTSQERSDVSRGQSGSRSVSRQTRNEKQSGDGSRHSGSRHHEASSRADSSRHSQVGQGQSSGPRTSRNQGSSVSQDSDSQGHSEDSERWSGSASRNHLGSAWEQSRDGSRHPGSHHEDRAGHGHSADSSRQSGTRHTESSSRGQAASSHEQARSSAGERHGSHHQLQSADSSRHSGIGHGQASSAVRDSGHRGYSGSQASDSEGHSEDSDTQSVSAQGKAGPHQQSHKESARGQSGESSGRSGSFLYQVSTHEQSESTHGQSAPSTGGRQGSHYDQAQDSSRHSASQEGQDTIRGHPGPSRGGRQGSHQEQSVDRSGHSGSHHSHTTSQGRSDASRGQSGSRSASRKTYDKEQSGDGSRHSGSHHHEASSWADSSRHSLVGQGQSSGPRTSRPRGSSVSQDSDSEGHSEDSERRSGSASRNHHGSAQEQSRDGSRHPRSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLYQVSTHEQSESAHGRTGTSTGGRQGSHHKQARDSSRHSTSQEGQDTIHGHPGSSSGGRQGSHYEQLVDRSGHSGSHHSHTTSQGRSDASHGHSGSRSASRQTRNDEQSGDGSRHSGSRHHEASSRADSSGHSQVGQGQSEGPRTSRNWGSSFSQDSDSQGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQYGQDTIRGHPGSSRGGRQGYHHEHSVDSSGHSGSHHSHTTSQGRSDASRGQSGSRSASRTTRNEEQSGDSSRHSVSRHHEASTHADISRHSQAVQGQSEGSRRSRRQGSSVSQDSDSEGHSEDSERWSGSASRNHRGSVQEQSRHGSRHPRSHHEDRAGHGHSADRSRQSGTRHAETSSGGQAASSHEQARSSPGERHGSRHQQSADSSRHSGIPRGQASSAVRDSRHWGSSGSQASDSEGHSEESDTQSVSGHGQAGPHQQSHQESARDRSGGRSGRSGSFLYQVSTHEQSESAHGRTRTSTGRRQGSHHEQARDSSRHSASQEGQDTIRGHPGSSRRGRQGSHYEQSVDRSGHSGSHHSHTTSQGRSDASRGQSGSRSASRQTRNDEQSGDGSRHSWSHHHEASTQADSSRHSQSGQGQSAGPRTSRNQGSSVSQDSDSQGHSEDSERWSGSASRNHRGSAQEQSRDGSRHPTSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLYQVSTHEQSESAHGRAGPSTGGRQGSRHEQARDSSRHSASQEGQDTIRGHPGSRRGGRQGSYHEQSVDRSGHSGSHHSHTTSQGRSDASHGQSGSRSASRETRNEEQSGDGSRHSGSRHHEASTQADSSRHSQSGQGESAGSRRSRRQGSSVSQDSDSEAYPEDSERRSESASRNHHGSSREQSRDGSRHPGSSHRDTASHVQSSPVQSDSSTAKEHGHFSSLSQDSAYHSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

SEQ ID NO: 2 - protein, 4061 aa, Homo sapiens; identical to SEQ ID NO: 1

SEQ ID NO: 3 - protein, 4061 aa, Homo sapiens; identical to SEQ ID NO: 1

SEQ ID NO: 4 - protein, 4061 aa, Homo sapiens; identical to SEQ ID NO: 1

SEQ ID NO: 5 - protein, 4061 aa, Homo sapiens; identical to SEQ ID NO: 1

SEQ ID NO: 6 - protein, 554 aa, Mus musculus
MSALLESITSMIEIFQQYSTSDKEEETLSKEELKELLEGQLQAVLKNPDDQDIAEVFMQMLDVDHDDKLDFAEYLLLVLKLAKAYYEASKNESFQTHGSNGRSKTDYKGLEEEGEEGNEQNLRRRHGGTDGKRKSDRTRSPNGKRGKRQESRCRSEGKDKHRREPEKHRHQQDSKRKQRHGSGSTERKDNRNKKNRQSKERNYDEIYDNGKYNEDWEASYNNCYYKTQNTTLDQREGNRRPRADSQKEPQSFHGQADNSDSEGGRQQSHSKPSPVRADQRRSRAGQAGSSKVSARSGSGGRGQSPDGSGRSSNRRDRPRQPSPSQSSDSQVHSGVQVEGRRGQSSSANRRAGSSSGSGVQGASAGGLAADASRRTGALQGQASAQGRAGSQGQAQGRVGSSADRQGRRGVSESQASDSEGQSDFSEGQAVGAHRQSGAGQRHEQRSSRGQYGSGFYPVYYYYEQEHSEEESDSQHGHQHEQQRGHQHQHQHEHEQPESGHRQQQSSGRGHQGAHQEQGRDSARSRGSNQGHSSSRHQADSPRVSVRSGSGGRGQ

SEQ ID NO: 7 - protein, 336 aa, Mus musculus
PDGSGRSSNRRDRPRQLSPSQSSDSQVHSGVQVEGRRGHSSSANRRAGSSSGSGVQGASAGGLAADASRRSGARQGQASAQGRAGSQGQAQGRVSSSADRQGRRGVSESRASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARPRGSNQGHSSSRHQADSPRVSARSGSGGRGQSPDASGRSSNRRDRPRQPSPSQSSDSQVHSGVQVEAQRGQSSSANRRAGSSSGSGVQGAAASGQGGYESIFTAKHLDFNQSHSYYYY

SEQ ID NO: 8 - protein, 250 aa, Mus musculus
GGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSSSGVQGASA

SEQ ID NO: 9 - protein, 255 aa, Mus musculus
GGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASA

SEQ ID NO: 10 - protein, 4838 aa, Mus musculus
MSALLESITSMIEIFQQYSTSDKEEETLSKEELKELLEGQLQAVLKIIQLLDDYPKCFIVGADNVGSKQMQQIRMSLRGKAVVLMGKKTMMRKAIRGHLENNPALEKLLPHIRGNVGFVFTKEDLTEIRAMLLANKVPAAARVGAIAPCEVTMPAQNTGLGPEKISFFQALGITTRISRGTIEILSDVQLIKTGDKVGVSEATLLNMLNISPSSFGLIIQQVFDNGSIYNPEVLDITEQALHSRFLEGVHNVASVCLQIGYLTVDSVLHSFINGYKRVLALSVETEYTFPLAEKVKAFLANPSAFATAAITAAPAAATAPAKPENPDDQDIAEVFMQMLDVDHDDKLDFAEYLLLVLKLAKAYYEASKNESFQTHGSNGRSKTDYKGLEEEGEEGNKQNLRRRHGGTDGKRKSDRTRSPNGKRGKRQESRCRSEGKDKHRREPEKHRHQQDSKRKQRHGSGSTERKDNRNKKNRQSKERNYDEIYDNGKYNEDWEASYNNCYYKTQNTTLDQREGNRRPRADSQKEPQSSHGQADNSDSEGGRQQSHSKPSPVRADQRRSRAGQAGSSKVSARSGSGGRGQSPDGSGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVILESRSKAGAGSPHLPTGGPDPAPAQGSRGPLQVDWQLTPPGALGRVKARHLPRVAQGHKAKHRAASARQLTGKGVEGSARVRPATARATLTSQKARQQQQSSGRGNQGAHQEQGRDSARSRGSNQGHSSSRHQADSPRVSARSGSGGRGQSPDASGRSSNRRDRPRQPSPSQSSDSHVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSRYYYEQEHSEEESDSQHQHGHQHEQQRGHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRIGSSADRQGRRGVSESQASDSEGHSDFSEGQAVGAHRQSEAGQRHEQRSSRGQHGSGYYYEQEHSEEESDSQHQHGHQHEQQRGTQHQHEHQQPESGHRQQQSSGRGHQGTHQEQGRDSARSRGSNQGHSSSRHQADSPRVSARSGSGGRGQSPDASGRSSNRRDRPRQPSPSQSSDSQVHSGVQVEGRRRQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGNQGAHQEQGRDSARSRGSNQGHSSSRHQADSPRVSARSGSGGRGQSPDASGRSSNRRDRPRQPSPSQSSDSHVHSGVQVEGRRGQSSSANRRAGSSSSSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASVRSSNRRDRPRQPSPSQSSDSHVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHKHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSSSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEEQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGTSAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGNQGAHQKQGRDSARSRGSNQGHSSSRHQADSPRVSARSGSGGRGQSPDASGRSSNRRDRPRQPSPSQSSDSHVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSGSQASDSEGHSDFSEGQAVGAHRQSGAGQRHEQRSSRGQHGSGYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSSSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQQGHQHEQQRGHQHQHQHQHEHEQPESGHRQQQSSGRGHQGAHQEQGRDSARSRGSNQGHSSSRHQADSPRVSARSGSGGRGQSPDGSGRSSNRRDRPRQPSASQSSDSQVHSGVQVEAQRGQSSSANRRAGSSSGSGVQSAAASGQGGYESIFTAKHLDFNQSHSYYYY

SEQ ID NO: 11 - protein, 336 aa, Mus musculus; identical to SEQ ID NO: 7

SEQ ID NO: 12 - protein, 357 aa, Mus musculus
MSALLESITSMIEIFQQYSTSDKEEETLSKEELKELLEGQLQAVLKNPDDQDIAEVFMQMLDVDHDDKLDFAEYLLLVLKLAKAYYEASKNESFQTHGSNGRSKTDYKGLEEEGEEGNKQNLRRRHGGTDGKRKSDRTRSPNGKRGKRQESRCRSEGKDKHRREPEKHRHQQDSKRKQRHGSGSTERKDNRNKKNRQSKERNYDEIYDNGKYNEDWEASYNNCYYKTQNTTLDQREGNRRPRADSQKEPQSSHGQADNSDSEGGRQQSHSKPSPVRADQRRSRAGQAGSSKVSARSGSGGRGQSPDGSGRSSNRRDRPRQPSPSQSSDSQVHSGVQVEGRRGQSSSANRRAGSSSGS

SEQ ID NO: 13 - protein, 687 aa, Rattus norvegicus
MNRNENMNTNSQSPATGSRGPVGERTGQLAMNRKATAPGKGALTRRTAPLAPRRRSHAEGLEPVPVRVRGPRGRLPGTALSTPSPEGARPSAVDAQDTPLGEPTKTATSRQQTALPAPGPGEARPAPTVRARPPVVRPAAEALQPPGTHLAPNSLQTLLAGPDPAVDSSPPNDMLTQPRAAQEPAPEEEESPQLASSPPTEPGTSNPGGVEPGRPAPASPVTAKAILEPMPGSGKGRPQPLTGGQAPAQGASGPLLVAKQLTPPVAQGPVKARHQARVVQGPEAGKLRASLVMGSRQTATGTLGPGKHKPTQPQALTAGGLAGPARVRPATARATPTTQKHTHRGAHGGIQTSQRHEQRPSRGQQGSGHPQVYYYGVEETEDESDAQQGHHQQQQQQRQQQRQRHQHEQEREHEHQQPESSHRQQGSSGRTHRAARHEQESDSTRQRGSHQAHSSARTQEEIARGRSGASASEGPGPQREAARDSSEHAQSRRSETISRGRSGHSTGRAHEDRHEQATDRSARSGSRGGQAGSHSESEASGGQAGRRGTAATRHTSRPEQSPDTAGRTGSSRGQQSAQRHADSTPGSTRTGSRGRGESPAGQQSPDRARHIESRRGRTREASASQSSDSEGHSGAHAGIGQGQTSTTHRRAGSSSGSQQASVEGSDGYDSIVKSKYLSFSQSHCYYY

SEQ ID NO: 14 - protein, 3088 aa, Pan troglodytes
MSTLLENIFAIINLFKQYSKKDKNTDTLSKKELKELLEKEFRQILKNPDDPDMVDVFMDHLDIDHNKKIDFTEFLLMVFKLAQAYYESTRKENLPISGHKHRKHSHHDKHEDNKQEENKENRKRPSSLERRNNRKGNKGRSKSPRETGGKRHESSSEKKERKGYSPTHREEEYGKNHHNSSKKQKNKTENTRLEDNRKRLSERLEEKEDNEEGGYDYENTGRMTQKWIQSGHIATYYTIQDEAYDTTDNLLEENKIYERSRSSDDKSSSQVNRSRHENTSQVPLQESRTRKRRGSRVSQDRDSEGHSEDSERHSGSASRNHPGSARQQSRDGSRHPRSHDEDRASHGHSADSSRQSGTRHAETSSRGQTASSHEQARSSPGERHGSRHQQSADSSRHSATGRGQASSAVSDRGPRGSSGSQASDSEGHSENSDTQSVSGHGKAGPRQQSHQESARGRSGERSGHSGSFLYQVSTHEQSDSAHGRTGTSTGGRQGSHHEQARDSSRHSASQEGQDTIRGHPGSSRGGRQGSHHEQLVNRSGHSGSHHSHTTSQGRSDASHGQSGSRSASRQTRNEEQSGDGSRHSGSRHHEASSQADSSRHSQVGQGQSSGPRTSSHQGSSVSQDSDSEGHSEDSERWSGSASRNHHGSAREQSRDGSRHPRSHHEDRAGHGHSADSSRQSGTRHTESSSRGQAVSSHEQARSSPGERHGSRHQQSADSSRHSGIGHGQASSAVRDSGHRGSSGSQAIDSEGHSEDSDTQSVSSHGQAGPHQQSHKESARGQSGESSGRSGSFLYQVSSHEQSESTYGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRASSSRHSQVGQGESAVSKTSRRQGSSVSQDRDSEGHSEDSERQSESASRNHYGSAREQSRHGSRNPRSHQEDRASHGHSAESSRQSGTHHAETSSHGQAASSQEQARSSRGERHGSRHQQSADSSTDSGTGGRQASSVVGDSGNRGSSGSQASDSEGHSEDSDTQSVSAHGQAGPRQQSHQESTRGQSGERSGRSGSFLYQVSTHEQSESAHGRTGPSTGGRQRSRHEQARDSSRHSASQESQDIIHAHPGSSRGGRQGSHYEQSVDRSGHSGSHHSHTTSQERSNASHGQSGSRSASRQTRNEEQSGDGSRHSGSRHHEASSRADSSRHSQVGQGQSSGPRTSRNQGSSVSQDSDSQGHSEDSERWSGSASRNHHGSAWEQSRDGSRHPGSHQEDRAGHGHSADSSRQSGTHHTESSSRGQAASSHEHARSSAGERHGSHHQQSADSSRHSGIGHGQASSAVRDSGHRGSSGSQASDSEGHSEDSDTQSLSAHGQAGPHQQSHQESTRGRSAERSGRSGSFLYQVSTHEQSESAHGRTGTSTGGRKRSLHEQARDSSRHSVSQEGQDTIHGHAGSSSGGRQGSHYEQLVDRSGHSGSHHSHTTSQGRSDAYHGQSGSRSASRQTHNDEQSGDGFRHSGSHHHEASSRADSSRHSQVGQGQSEGPRTSRHRESSVSQDSDSEGHSEDSERWSGSASRNHHGSAREQSRDGSRHPRSHHEDRAGHGHSADSSRQSGTRHTQTSSGGQAASSHEQGRSSAGERHGSRHQQSADSSRHSGIGHGQASSAVRDSGHRGYSGSQASDSEGHSEDSDTQSVSAHGQAGSHQQSHQESARGRSGERSGRSGSFLYQVSTHEQSESSHGWTGPSTRGRQGSRHEQAQDSSRHSASQEGQDTIRGHPGPSRGGRQGYHHEQSVDSSGHSGSHHSHITSQGSSHASHGQSGSRSASRTTRNDEQSVDGSRHSGSRHHEASTHADISRHSQAGQGQSEGSRTSRRQGSSVSQDSDSEGHSEDSERWSGSASRNHRGSAQEQSRDGSRHPRSHHEDRAGHGHSADSSRQSGTHHAETSSGGQAASSREQARSSPGERHGSRHQQSADSSRHSGIRRGQASSAVRDSGHWGSSGSQASDSEGHSEESDTQSVSGHGQDGPHQQSHQESARDRSGGRSGRSGSFLYQVSTHEQSESTHGQTGTSTGGRQGSHHEQARDSSRHSASQEGQDTIHAHPGSSRGGRQGSHHEQSVDTSGHSGSHHSHTTSQGRSDASHGQSGSRSASRQTRNDEQSGDGSRHSGSHHHEAFTQADSSRHSQSGQGESAGSRRSRRQGSSVSQDSDSQGHSEDSERWSGSASRNQHGSAREQSRDGSRHPGSHQEDRAGHGHSADSSRQSGTRHTESSSRGQAASSHEQARSSAGERHGSRHQLQSADSSRHSGIGHGQASSAVRDSGHRGSSGSQAIDSEGHSEDSDTQSVSAQGQAGPHQRSHKESARGQSGESSGRSGSFLYQVSTHEQPESTHGQSAPSTGGRQGSHHDQAQDSSRHSASQEGQDTIRGHPGPSRGGRQGSHHKQSVDRSGHSGSHHSHTTSQGRSDASRGQSGSRSASRQTHDKEQSGDGSRHSGSRHHEASSWADSSRHSQAGQGQSEGSRTSRRQGSSFSQDSDSEGHSEDSERRSGSASRNHRGSAQEQSRDGSRHPRSHHEDRAGHGHSADSSRQSGTHHAQNSSGGQAASFHEQARSSAGERHGSHHQQSADSSRHSGIGHGQASSAVRDSGHRGSSGSQASDSEGHSEDSDTQSVSAHGQAGPHQQSHQESTRGRSAERSGRSGSFLYQVSTHEQSESAHGRTGPSTGGRQGSRHEQARDSSRHSASQEGQDTIHGHPGSRRGGRQGSYHEQSVDRSGHSGSHHSHTTSQGRSDASHGQSGSRSASRQTRNEQQSGDGSRHSGSSHHEASTQADSSRHSQSGQGESAGSRRSRRQGSSVSQDSDSEAYPEDSERRSESASRNRHGSSREQSRDGSRHPGSSHRDTTRHVQSSPVQSDSSTAKEHGHFSSLSQDSAYRSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

SEQ ID NO: 15 - protein, 2764 aa, Pan troglodytes
MSTLLENIFAIINLFKQYSKKDKNTDTLSKKELKELLEKEFRQILKNPDDPDMVDVFMDHLDIDHNKKIDFTEFLLMVFKLAQAYYESTRKENLPISGHKHRKHSHHDKHEDNKQEENKENRKRPSSLERRNNRKGNKGRSKSPRETGGKRHESSSEKKERKGYSPTHREEEYGKNHHNSSKKQKNKTENTRLEDNRKRLSERLEEKEDNEEGGYDYENTGRMTQKWIQSGHIATYYTIQDEAYDTTDNLLEENKIYERSRSSDDKSSSQVNRSRHENTSQVPLQESRTRKRRGSRVSQDRDSEGHSEDSERHSGSASRNHPGSARQQSRDGSRHPRSHDEDRASHGHSADSSRQSGTRHAETSSRGQTASSHEQARSSPGERHGSRHQQSADSSRHSATGRGQASSAVSDRGPRGSSGSQASDSEGHSENSDTQSVSGHGKAGPRQQSHQESARGRSGERSGHSGSFLYQVSTHEQSDSAHGRTGTSTGGRQGSHHEQARDSSRHSASQEGQDTIRGHPGSSRGGRQGSHHEQLVNRSGHSGSHHSHTTSQGRSDASHGQSGSRSASRQTRNEEQSGDGSRHSGSRHHEASSQADSSRHSQVGQGQSSGPRTSSHQGSSVSQDSDSEGHSEDSERWSGSASRNHHGSAREQSRDGSRHPRSHHEDRAGHGHSADSSRQSGTRHTESSSRGQAVSSHEQARSSPGERHGSRHQQSADSSRHSGIGHGQASSAVRDSGHRGSSGSQAIDSEGHSEDSDTQSVSSHGQAGPHQQSHKESARGQSGESSGRSGSFLYQVSSHEQSESTYGQTAPSTGGRQGSRHEQARNSSRHSASQDGQDTIRGHPGSSRGGRQGSYHEQSVDRSGHSGYHHSHTTPQGRSNASHGQSGSRSASRQTRNEEQSGDGSRHSGSRHHEASSRADSSRHSQVGQGQSSGPRTSRNQGSSVSQDSDSQGHSEDSERWSGSASRNHHGSAWEQSRDGSRHPGSHQEDRAGHGHSADSSRQSGTHHTESSSRGQAASSHEHARSSAGERHGSHHQQSADSSRHSGIGHGQASSAVRDSGHRGSSGSQASDSEGHSEDSDTQSLSAHGQAGPHQQSHQESTRGRSAERSGRSGSFLYQVSTHEQSESAHGRTGTSTGGRKRSLHEQARDSSRHSVSQEGQDTIHGHAGSSSGGRQGSHYEQLVDRSGHSGSHHSHTTSQGRSDAYHGQSGSRSASRQTHNDEQSGDGFRHSGSHHHEASSRADSSRHSQVGQGQSEGPRTSRHRESSVSQDSDSEGHSEDSERWSGSASRNHHGSAREQSRDGSRHPRSHHEDRAGHGHSADSSRQSGTRHTQTSSGGQAASSHEQGRSSAGERHGSRHQQSADSSRHSGIGHGQASSAVRDSGHRGYSGSQASDSEGHSEDSDTQSVSAHGQAGSHQQSHQESARGRSGERSGRSGSFLYQVSTHEQSESSHGWTGPSTRGRQGSRHEQAQDSSRHSASQEGQDTIRGHPGPSRGGRQGYHHEQSVDSSGHSGSHHSHITSQGSSHASHGQSGSRSASRTTRNDEQSVDGSRHSGSRHHEASTHADISRHSQAGQGQSEGSRTSRRQGSSVSQDSDSEGHSEDSERWSGSASRNHRGSAQEQSRDGSRHPRSHHEDRAGHGHSADSSRQSGTHHAETSSGGQAASSREQARSSPGERHGSRHQQSADSSRHSGIRRGQASSAVRDSGHWGSSGSQASDSEGHSEESDTQSVSGHGQDGPHQQSHQESARDRSGGRSGRSGSFLYQVSTHEQSESTHGQTGTSTGGRQGSHHEQARDSSRHSASQEGQDTIHAHPGSSRGGRQGSHHEQSVDTSGHSGSHHSHTTSQGRSDASHGQSGSRSASRQTRNDEQSGDGSRHSGSHHHEAFTQADSSRHSQSGQGESAGSRRSRRQGSSVSQDSDSQGHSEDSERWSGSASRNQHGSAREQSRDGSRHPGSHQEDRAGHGHSADSSRQSGTRHTESSSRGQAASSHEQARSSAGERHGSRHQLQSADSSRHSGIGHGQASSAVRDSGHRGSSGSQAIDSEGHSEDSDTQSVSAQGQAGPHQRSHKESARGQSGESSGRSGSFLYQVSTHEQPESTHGQSAPSTGGRQGSHHDQAQDSSRHSASQEGQDTIRGHPGPSRGGRQGSHHKQSVDRSGHSGSHHSHTTSQGRSDASRGQSGSRSASRQTHDKEQSGDGSRHSGSRHHEASSWADSSRHSQAGQGQSEGSRTSRRQGSSFSQDSDSEGHSEDSERRSGSASRNHRGSAQEQSRDGSRHPRSHHEDRAGHGHSADSSRQSGTHHAQNSSGGQAASFHEQARSSAGERHGSHHQQSADSSRHSGIGHGQASSAVRDSGHRGSSGSQASDSEGHSEDSDTQSVSAHGQAGPHQQSHQESTRGRSAERSGRSGSFLYQVSTHEQSESAHGRTGPSTGGRQGSRHEQARDSSRHSASQEGQDTIHGHPGSRRGGRQGSYHEQSVDRSGHSGSHHSHTTSQGRSDASHGQSGSRSASRQTRNEQQSGDGSRHSGSSHHEASTQADSSRHSQSGQGESAGSRRSRRQGSSVSQDSDSEAYPEDSERRSESASRNRHGSSREQSRDGSRHPGSSHRDTTRHVQSSPVQSDSSTAKEHGHFSSLSQDSAYRSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

SEQ ID NO: 16 - protein, 1183 aa, Macaca mulatta
MSTLLENIFAIINLFKQYSKKDKNTDTLSKKELKELLEKEFRPILKNPDDPDTVEVFMDHLDIDHNKKIDFTEFLLMVFKLAQAYYESTRKQNLPIAGHKHRKHSHHDKHEDNKEEENKEKRKRPLSLERRNNRKGNTGRSKSPRERGGKRHESSFEKKERKGYSPTYEEEEYGQNHHKSSKKEKNKTEITRLEHEGKRISERPEKKEEKEDGQFDYENAGRMDEKWTESGHIAIYHAIQDEVDDTTENILEENRRYETSRSPHDKSSLRVNRSPNANTSQIPLVEPRRRTRQRSSVSQDSDSEGHSEDSERQSESASRNHHGSVREQSRHGSRHPGSHHEDRAGHGHSADSSTQSGTRHTETSSRGQAVSSHEQARSSPGERHGSRHQQSAESSRHSGIGRGQASSAVSDRGHQGPSGSHFSDSEGHSEHSDTQSVSGHGQAGPHPHSHQESARGRSRERSGRSGSFLYQVSTHEQSESTHGRTGPSSAGRQGSRNEQARDSSRHSASHEVQDTVHGHHGSSRGRRQGSHHEQLVDSSGHSGSHHSHTTSQGRSDASRGESGARSASRQTRHEEQSGDGSRHSGSRHHESSNRADSSRHAQSSQGQSAGFRTSTRRGSSVSQDSDSEGHSEDSERQSESASRNHHGSVREQSRHGSGHSGSHHQDKVGHRYSGDSSRQSGTHHVETSSHGQAASSHEQTRSSPGERHGSHHQQSADSSRHSGTGRGQASSAVSDRGHQGPSGSHFSDSEGHSEHSDTQSVSGQGQAGRHPHSHQESARGRSGERSGRSGSFVYQVSTHEQSESTHGRTGPSTGGRQGSRNEQARDSSRHSVSHEGQDTIHGHHGSSRGGRQGSHHEQSVDSSGHSGSHHSHTTSQGRSDASRGESGARSASRQTRHEEQSGDGSRHSGSRHHEASNRADSSRHAQSGQGQSAGFRTSTRRGSSVSQDSDSEGHSEDSERQSESASRNHHGSVREQSRHGSRHPGSHHEDRAGHGHSADSSRQSGTRHTETSSRGQAVSSHEQARSSPGERHGSRHQQSAESSRHSGIGRGQASSAVSDRGHQGPSGSHFSDSEGHSEHSDTQSVSGHGQAGPHPHSHQESARGRSGERSGRSGSFLYQVSTHEQSESTHGRTGPSSAGRQGSRNEQARDSSRHSASHEVQDTVHGHHGSSRGGRQGSHHEQS

SEQ ID NO: 17 - protein, 2949 aa, Macaca mulatta
  REGION 2160..2164: note = misc_feature - Xaa can be any naturally occurring amino acid
MTDLLRSVVTVIDVFYKYTKQDGECGTLSKDELKELLEKEFHPVLKNPDDPDTVDVIMHMLDRDHDRRLDFTEFLLMIFKLTMACNKVLSKEYCKASGSKKHRRGHRHQEEESETEEDEEDTPEHKSGYRHSSWSEGEEHGYSSGHSRGTVKRRHGSNSRRLGRQGHLSSSGNQERSQKRYHRSSSGHSWSSGKERHGFSSGELRERINKSHVSPSREFGEEYESGSGSKSWERKGHGGLSCGLEISGHESNSTQSRSSGQKLGSSRSCSGDSRRRSHACGYSNSSGCGRPQNASNSCQHRFGGQVNQSSYIQSGCQSGINGEQGHDCVSGGQPSGCGQPESNSCSQSYSQRGYGARENGQPQNCGGQQRTGSSQSSFCGQYESGGSQSCSNGQHEHGSCGRFSNSSSSNEFSKCGKHRSGSGQFTSCEQHGTGLSQSSGFEQQVAGSSQTCSQYGSRSSQSSGYDEHGSSSGKTSGFGQHRSGSGHSSGFGQHGSGSGQSFGFGQHGSGSGQSSGFGQHESRSCQSSYGQHGSGSSQSSGYGQHASRQTSGFGQHGLGSGQSTGFGQYGSGSGQSSGFGQHGSGSGQSSGFGQHESRSGQSSYGQHSSSSSQSSGYGQHGSRQTSGFGQHGSGSGQSTGFGQYGSSLGQSSGFGQHGSGSGQSSGFGQHESTSGQSSYGQHGFGSSQSSGCGQHGLSSGQTSGFGQHELSSGQSSSFGQHGSGSGQSSGFRQHESGSGQSSGFGQHESRSHQSSYGPHGSGSGQSSGYGQHGSSSGQTSGFGQQGSSSSQYSGFGQHGSGLGQSSGFGQHGTGSGQFSGFGQHESRSHQSSYGQHGSGSSQSSGYGQHGSSSGHTTGFGQHRSSSGQYSGFGQHGSGLGQSSGFGQHGTGSGQSSFGQHESRSHQSSYGQHGSGSSQSSSYGQHGSSSGQTSGFGQHRSGSGQSSGFGQYGLGSGSGFGQHGSGTGQSSGFARHEYRSGQSSYGQHGTGSSQSSGCGQRESGSGPTTGFGQHVSGSDNFSSSGQHISGSDQSTGFGQYGSGSGQSTGLGQVESQQVASGSTVHGRQETTHGQTINTARHSQSGQGQSTQTGSRVSRRRRSSQSENIDSEVHSRVSHRHSEHIDTQVGSHYPESGSTVHRRQGTTHGQRGDTTRHSHSGHGQSTQTGSRTTGRQRFSHSDATDSEVHSGVSHRPHSQEHTHGQDGSQLGESQSTVHERHETTYGQTGDATGHGYSGHGQSTQIGSRTSGRRGSGHSESSDTEVHSGGSHRPHSQEQTHGQARSQHGESRSTVHERHGTTHGQTGDTTRYAHYHNGQSAQRGSRTTGRGSGHSEYSDSELYSGGSHTYSGHTHGQAGSQHGESDSIVHERYGTTHGQTGDTTRHAHYSHGQSKQRGSRTTGRRGSGHSEYSDSEGHSGGSHTHSGHTHGHTHGQAGSQHGESGSSGHGGQGTTHGQTGDTTRHAHYGHGQSTQRGSRTAGRRGSGHSEYSDSEGHSGVSHTHSGHTHGQAGSQHGESESTVHERQQTTHGQTGDTTRHAHYGHGQSTQTGSRTTGRRGSGHSEYSDSEGHSGVSHTHSGHTHGQARSQHGESGSAIHGRQGTIHGQTGDTTRHGQSGHGQSTQTGSRTTGRRGSGHSEYSDSEGHSGGSHTHSGHTHGQAGSQHGESGSTVHGRQGTIHGQTGDTTRHGQSGHGQSIETGSRTTGRRGSGHSEYSDSEGHSGVSHTHSGHTHGQAGSQHGESESTVHERQQTTHGQTGDITEHGHSSHGQTTQTGSRTTGRRGSGHSEYSDSEWHSGSHTHSGHTHGQAGFQHGESGSAVHGRQGTIHGQTGDTTRHGQSGHGESIQTGSRTIGRRGSGHSEYSDSEGHSGISHTHSGHTHGQAGSQHGESGSSGHGRQGTAHGQTGDTTRHAHYDHGQSTQRGSRTAGRRGSGHSEYSDSEGHSGVSHTHSGHTHGQAGSQHGESGAAVHGRQGIIHGQTGDTTRHGQSGQGQSTQRGSRTTGRRGSGHSEYSDSVGHSGVSHTHSGHTHGLAGSQHGESGSSGHGRQGTLHGQTGDTTRHAHYGHGQSTQRGSRTAGRRGSGHSEYSDSEWHSGGSHTHSGHTHGQAGSQHGESGSAVHGRQGTIHGQTGDTTRHGQSGHGQSTQIGSRTTGRRGSGHXXXXXRHTETSSRGQAVSSHEQARSSPGERHGSRHQQSAESSRHSGIGRGQASSAVSERGHQGPSGSLFSDSEGHSEHSDTQSVSGHGQAGPHPHSHQESARGRSGERSGRSGSFLYQVSTHEQSESTHGWTGPSSAGRQGSRNEQARDSSRHSVSHEVQDTVHGHSGSSRGRRQGSHHEQLVDSSGHSGSHHSHTTSQGRSDASRGESGARSASRQTRHEEQSGDGSRHSGSRHHEASNRADSSRHAQSGQGQSAGFRTSTRRGSSVSQDSDSEGHSEDSERQSESASRNHHGSVREQSRHGSRYPGSHHEDGAGHGHTADSPRQSGTHHVETSSHGQAASSHEQTRSSPGERHGSRHQQSAESSRHSGIGRGQASSAVSERGHQGPSGSQFSDSEGHSEHSDTQSVSGHGQAGPHPHSHQESARGRSGERSGRSGSFVYQVSTHEQSESTHGQTGPSTGGRQGSRNEQARDSSRHSVSHEGQDTIHGHSESSIGGRQGSHHEQSVDSSGHSGSHHSHTTSQGRSDASRGQSGSRSASRQTRHEEQSGDGYRHSGSRLHEASTQADSFRHSHSGQGQSAGSRRSTLRGSSVNQDSASEGHSEDSERQSESASRNPHGAVREQSRDVSRHPRSYHHDTGNHVQSSSVYSDTTTSKEHGHFGSLSQDSAYHSGIQSRGSPHSSSSYNYHSEGTERERGQSGLVWRHGSYGSADYDYGESRFRHSQHGSVSYNSNPVVFKERSDIRKASAFGEDHPRYYARYVNRQPGLYRHSSDISKQLGFSQSQRYYYYE

SEQ ID NO: 18 - protein, 25 aa, Staphylococcus epidermidis
MKKLAFAITAASGAAAVLSHHDAEA

SEQ ID NO: 19 - protein, 30 aa, Staphylococcus epidermidis
WLDNRAFSKKFVPVVMATSVALFFLNLAFA

SEQ ID NO: 20 - protein, 29 aa, Staphylococcus epidermidis
MAKKFNYKLPSMVALTLFGTAFTAHQANA

SEQ ID NO: 21 - protein, 27 aa, Staphylococcus epidermidis
MKKRFLSICTMTIAALATTTMVNTSYA

SEQ ID NO: 22 - protein, 30 aa, Staphylococcus epidermidis
NLKKQSKLILIFICIFTFFIMIIQSQFLMG

SEQ ID NO: 23 - protein, 26 aa, Staphylococcus epidermidis
MKIFKLTSLTLAALTLAFPFSHVAQA

SEQ ID NO: 24 - protein, 28 aa, Staphylococcus epidermidis
MKKTVIASTLAVSLGIAGYGLSGHEAHA

SEQ ID NO: 25 - protein, 27 aa, Staphylococcus epidermidis
MKKNKFLVYLLSTALITPTFATQTAFA

SEQ ID NO: 26 - protein, 35 aa, Staphylococcus epidermidis
MKTRQNKYSIRKFSVGASSILIAALLFMGGGSAQA

SEQ ID NO: 27 - protein, 37 aa, Staphylococcus epidermidis
MKNNNETRRFSIRKYTVGVVSIITGITIFVSGQHAQA

SEQ ID NO: 28 - protein, 30 aa, Staphylococcus epidermidis
MKKKLSYMITIMLAFTLSLALGLFFNSAHA

SEQ ID NO: 29 - protein, 13 aa, Human immunodeficiency virus
GRKKRRQRRRPPQ

SEQ ID NO: 30 - protein, 27 aa, synthetic construct
GWTLNSAGYLLGKINLKALAALAKKIL

SEQ ID NO: 31 - protein, 18 aa, synthetic construct
KLALKLALKALKAALKLA

SEQ ID NO: 32 - protein, 30 aa, synthetic construct
WEAKLAKALAKALAKHLAKALAKALKACEA

SEQ ID NO: 33 - protein, 21 aa, synthetic construct
KETWWETWWTEWSQPKKKRKV

SEQ ID NO: 34 - protein, 9 aa, synthetic construct
RRRRRRRRR

SEQ ID NO: 35 - protein, 24 aa, synthetic construct
LGTYTQDFNKFHTFPQTAIGVGAP